(19) United States
(12) Patent Application Publication
(10) Pub. No.: US 2006/0183786 A1
(43) Pub. Date: Aug. 17, 2006

(54) Injectable long-acting analgesic composition comprising an ester derivative of ketorolac

(75) Inventor: Jhi-Joung Wang, Tainan (TW)

Correspondence Address: FOLEY AND LARDNER LLP;SUITE 500, 3000 K STREET NW, WASHINGTON, DC, 20007, US

(73) Assignee: CHI MEI FOUNDATION HOSPITAL

(21) Appl. No.: 11/058,353

(22) Filed: Feb. 16, 2005

(51) Int. Cl. A61K 31/407

(52) U.S. Cl. 514/412

(57) ABSTRACT

Disclosed herein is an injectable long-acting analgesic composition comprising:
(a) a ketorolac ester derivative of formula (I), 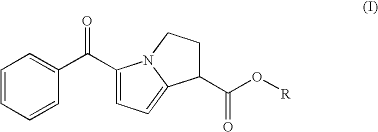 wherein
R is a straight-chain or branched saturated or unsaturated C₁-C₂₀ aliphatic group optionally substituted with a C₆-C₁₀ aryl group; and (b) a pharmaceutically acceptable oil vehicle. The composition can provide a longer duration of action and, therefore, is suitable for use in the treatment of long-lasting pains and inflammations.

### BACKGROUND OF THE INVENTION

1. Field of the Invention

This invention relates to an injectable long-acting analgesic composition comprising an ester derivative of ketorolac and a pharmaceutically acceptable oil vehicle.

2. Description of the Related Art

Most patients who experience moderate to severe pain, such as post-operative pain, post-traumatic pain and burn pain, often require pain control in the first 3 days after injury. An analgesic with a long-acting effect of around 3 days may be particularly valuable for this purpose (K.-S. Chu, et al. (2003), Anesthesia Analgesia, Vol. 97, 806-809). Currently, nonsteroidal anti-inflammatory drugs (NSAIDs) are often used in this field (J. C. Grillis et al. (1997), ADIS Drug Evaluation, Vol. 53, 139-188), but all of them are short-acting drugs. Prolonging the duration of action would make NSAIDs, e.g., a potent NSAID, more valuable in treating long-lasting pains clinically.

Amongst NSAIDs, ketorolac is the most potent one. Ketorolac, the chemical name of which is (±)-5-benzoyl-2,3-dihydro-1H-pyrrolizine-1-carboxylic acid or 5-benzoyl-1,2-dihydro-3H-pyrrolo[1,2-a]pyrrole-1-carboxylic acid, has a molecular weight of 255.27 and is represented by the following formula (A):

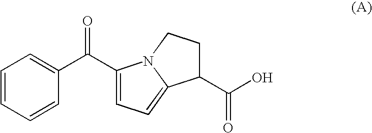

The major mechanism by which ketorolac and other NSAIDs exert their pharmacological effects is inhibition of prostaglandin synthesis. In particular, it is well believed that the primary action of ketorolac (as all NSAIDs) is to inhibit cyclooxygenase, which is responsible for the biosynthesis of prostaglandins, prostacyclin and thromboxane. Prostaglandins, which are released from virtually all tissues in response to direct trauma, act to mediate pain and inflammation.

Ketorolac has a very strong analgesic activity of opioid level (J. C. Gillis et al. (1997), supra). The analgesic efficacy of ketorolac has been extensively evaluated in the postoperative setting, in both hospital inpatients and outpatients, and in patients with various pain states. Intramuscular administration of 10-30 mg ketorolac can provide an analgesic efficacy similar to that of intramuscular administration of 6-12 mg morphine or 50-100 mg pethidine.

As a NSAID, ketorolac possesses analgesic, anti-inflammatory and antipyretic activities (M. M. T. Buckley et al. (1990), Drugs, Vol. 39, 86-109). Preoperative administration of ketorolac reduces pain in the immediate post-operative period (J. B. Forrest et al. (1997), Drug Safety, Vol. 16, 309-329). Combination therapy with ketorolac and opioids results in a significant 25% to 50% reduction in morphine and fentanyl requirements in the first 1 to 2 post-operative days, and may be accompanied by a reduction in opioid-induced adverse events. In addition, some patients experience a more rapid return to normal gastrointestinal function and shorter stay in hospitals.

Ketorolac is available for intramuscular, intravenous or oral administration, and is indicated for the short-term treatment of moderate to severe pain which requires analgesia at the opioid level. The usual parenteral dosage is 10-30 mg every 4 to 6 hours with a maximum total daily dose of 90 mg and a maximum duration of therapy of 5 days. For post-operative analgesia, single or multiple doses of intramuscular or intravenous administration of 10-30 mg ketorolac can provide an analgesic efficacy similar to that of intramuscular administration of 6-12 mg morphine, 50-100 mg pethidine or 30 mg pentazocine, or intravenous administration of 2-4 mg morphine, and greater than that of intramuscular administration of 75 mg diclofenac. The analgesic effect of ketorolac tends to be slower in onset than that of morphine or pethidine but persists for longer periods (M. M. T. Buckley et al. (1990), supra).

When administered via intravenous patient-controlled analgesia, 5 mg/h ketorolac provides pain relief similar to that of 1 mg/h morphine, 330 mg/h dipyrone (or called metamizole, metamisole) and 15 mg/h lysine clonixinate (or called clonixin-lysinate) in patients after major abdominal surgery, but may be less effective than 15 mg/h tramadol. Intravenous or intraarticular administration of ketorolac combined with bupivacaine or lidocaine (lignocaine) provides better analgesia than either agent alone in patients after knee arthroscopy or hand surgery. Subcutaneous administration of 60-120 mg/day ketorolac was beneficial in the treatment of some patients with cancer pain, especially those with a component of pain resulting from bone metastases, and was accompanied by a concomitant reduction in opioid dosage. However, in other cancer patients, morphine was more effective than ketorolac but less well tolerated (M. M. T. Buckley et al. (1990), supra).

A long-acting analgesic effect is particularly desirable in patients suffering from pain, such as post-operative pain, post-traumatic pain, and burn pain, which may last for around 3 days. Ketorolac is a NSAID which has a strong but short acting analgesic activity. Prolonging the duration of action would make ketorolac more valuable in the treatment of pain clinically.

Several ketorolac ester prodrugs have been synthesized and reported previously. However, to the applicant's knowledge, none of the known ketorolac esters was applied for a purpose of long-acting effect. For example, in Journal of Pharmaceutical Sciences (1994), Vol. 83 (11), 1548-1553, Samir D. Roy and Elizabeth Manoukian reported the permeability of ketorolac (in free acid form) and its two ester analogues, i.e. ketorolac [(N,N-dimethylamino) carbonyl]methyl ester (KDAE) and ketorolac ethyl ester (KEE), through human cadaver skin. KDAE was reported to be a better ester prodrug than KEE because it exhibited relatively higher skin flux and faster enzymatic hydrolysis by human serum to liberate the parent drug, i.e. ketorolac.

In addition, Carlos E. A. Monti et al. disclosed in U.S. Pat. No. 5,508,301 and U.S. Pat. No. 5,574,170 that the oxalate salt of 2-(1-pyrrolidinyl)ethylester of ketorolac and the maleate salt of 2-(diethylamino)ethyl ester of ketorolac (comparative compound) exhibited less undesired side effects (i.e. gastrointestinal irritation and ulceration) than the commercially available ketorolac (trometamole salt).

Likewise, to prevent gastrointestinal ulceration and acute renal failure caused by long-term use of ketorolac, H.-J. Doh et al. reported in Journal of Pharmaceutical Sciences (2003), Vol. 92 (5), 1008-1017, the synthesis and evaluation of several alkyl ester prodrugs of ketorolac, including ketorolac methyl, ethyl, isopropyl, 1-propyl, isobutyl, 1-butyl, and 1-pentyl esters, for transdermal delivery. They found that, amongst these ester prodrugs, the permeation rate of ketorolac in rat skin reached a maximum of 46.61 nmole/cm²/h in its 1-propyl ester form.

It is further noted that ketorolac tromethamine, which is represented by the following formula (B), 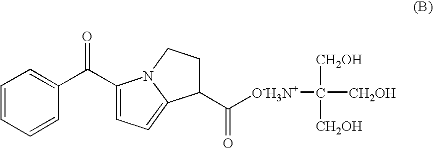
has been used clinically in three different dosage forms, i.e. injection (e.g., Toradol®), table/pill (e.g., Toradol®) and ophthalmic solution (drop)(e.g., Acular®). As an ophthalmic drug (drop), ketorolac tromethamine can be used to relieve eye itching caused by allergies. Ketorolac tromethamine in injection or tablet/pill form can be used to treat serious, short-term pains. However, like ketorolac, ketorolac tromethamine can only provide short-term (up to 5 days) management of severe, acute pains that require analgesia at the opiate level. It may cause severe side effects if taken for a longer time.

Accordingly, there is still a need in the art to develop a suitable pharmaceutical composition that allows ketorolac to exhibit a long-acting analgesic effect.

### SUMMARY OF THE INVENTION

Therefore, according to a first aspect, this invention provides an injectable long-acting analgesic composition comprising:

(a) a ketorolac ester derivative of formula (I): 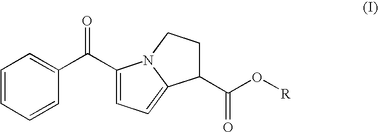
wherein
R is a straight-chain or branched saturated or unsaturated C₁-C₂₀ aliphatic group optionally substituted with a C₆-C₁₀ aryl group; and

(b) a pharmaceutically acceptable oil vehicle.

As compared to ketorolac or ketorolac tromethamine, the composition according to this invention can provide a longer duration of action and, therefore, is suitable for use in the treatment of long-lasting pains and inflammations.

Therefore, in the second aspect, this invention provides a method of providing a prolonged analgesia to a subject, including human and animal, comprising intramuscularly or subcutaneously administering an effective amount of the composition described above to a subject in need of such treatment.

In the third aspect, this invention provides a method of providing a prolonged anti-inflammatory effect to a subject, including human and animal, comprising intramuscularly or subcutaneously administering an effective amount of the composition described above to a subject in need of such treatment.

### BRIEF DESCRIPTION OF THE DRAWINGS

The above and other features and advantages of the present invention will become apparent in the following detailed description of the preferred embodiments with reference to the accompanying drawing, of which:

FIG. 1 shows the dose response study of the analgesic effect of ketorolac tromethamine (prepared in 0.9% saline) upon intramuscular injection to rats intraplantarly injected with carrageenin;

FIG. 2 shows the dose response study of the anti-inflammatory effect of ketorolac tromethamine (prepared in 0.9% saline) upon intramuscular injection to rats intraplantarly injected with carrageenin;

FIG. 3 shows the dose response study of the analgesic effect of ketorolac propyl ester (prepared in sesame oil) upon intramuscular injection to rats intraplantarly injected with carrageenin;

FIG. 4 shows the dose response studies of the anti-inflammatory effects of ketorolac propyl ester (prepared in sesame oil) upon intramuscular injection to rats intraplantarly injected with carrageenin;

FIGS. 5-11 show the analgesic effects of ketorolac and six ketorolac ester derivatives (all prepared in sesame oil) upon intramuscular injection to rats intraplantarly injected with carrageenin, respectively;

FIGS. 12-18 show the anti-inflammatory effects of ketorolac and six ketorolac ester derivatives (all prepared in sesame oil) upon intramuscular injection to rats intraplantarly injected with carrageenin, respectively;

FIGS. 19-22 show the analgesic effects of four different oil preparations of ketorolac propyl ester upon intramuscular injection to rats intraplantarly injected with carrageenin, respectively; and

FIGS. 23-26 show the anti-inflammatory effects of four different oil preparations of ketorolac propyl ester upon intramuscular injection to rats intraplantarly injected with carrageenin, respectively.

### DETAILED DESCRIPTION OF THE INVENTION

Ketorolac does not appear to directly activate mu (μ) or kappa (κ) opioid receptors. Ketorolac alone does not attenuate the response to visceral nociception in rats. However, when co-administered with morphine, it resulted in a marked potentiation of analgesia which could be completely reversed by naloxone. The mechanism by which ketorolac may exert a central effect is unclear but may include a modulatory effect on opioid receptors or alteration of opioid pharmacokinetics (M. M. T. Buckley et al. (1990), supra).

In contrast to opioid drugs, ketorolac does not alter gastric motility or haemodynamic variables or adversely affect respiration, nor is it associated with adverse CNS effects, abuse or addiction potential. The pharmacokinetics of ketorolac are linear over the usual oral and parenteral dosage range.

Parenteral administration of 10-60 mg ketorolac can provide effective pain relief in a majority of patients afflicted with acute pains caused by various conditions, including renal colic pain, sickle cell crisis, migraine, headache, fractures, strains, sprains and gout. As used in the post-operative setting, no significant difference in response to therapy has been seen amongst patients treated with ketorolac and morphine, pethidine, pentazocine, ibuprofen, diclofenac or indomethacin in standard doses (M. M. T. Buckley et al. (1990), supra).

In general, pre-, intra- or post-operative parenteral administration of a single dose of 30-60 mg ketorolac appears to be an effective alternative to parenterally administered opioid agents, such as 50-100 μg fentanyl, 100 mg pethidine, 6 mg dezocine, or other NSAIDs, such as 75-100 mg diclofenac (intramuscular or rectal), 100 mg indomethacin (rectal) and 40 mg piroxicam (oral), after various outpatient laparoscopic or orthopaedic procedures associated with mild to moderate pains (L. A. Smith et al. (2000) British Journal of Anaesthesia Vol. 84, 48-58).

The oral bioavailability of ketorolac is about 80% to 100%, and peak plasma concentrations (Cₘₐₓ) are achieved within about 30 to 60 minutes after oral or parenteral administration. As with other NSAIDs, ketorolac is almost entirely bound to plasma proteins (>99%), which results in a small apparent volume of distribution (Vd)(<0.3 L/kg). It is extensively metabolized, primarily by conjugation with glucuronic acid, and excreted via the kidney. Its metabolites have no significant analgesic activity. The mean terminal elimination half-life (T_{1/2β}) of ketorolac in healthy volunteers is about 5 hours. In the elderly, while the absorption and plasma protein binding of ketorolac are unaffected, plasma drug clearance (CL) is reduced, which results in a moderate prolongation of T_{1/2β} to about 6 to 7 hours. As would be expected in patients with renal impairment, the plasma clearance of ketorolac is decreased, which results in an increased T_{1/2β} (9 to 10 hours). Slight increases in T_{1/2β} and time to Cₘₐₓ are seen in patients with alcoholic cirrhosis (M. M. T. Buckley et al. (1990), supra).

Most adverse events associated with ketorolac involve the gastrointestinal tract and range from mild upset to serious ulceration and hemorrhage. Results from a large post-marketing surveillance study (n>20,000) indicate that, overall, parenteral ketorolac is associated with only a slightly increased risk of gastrointestinal or operative site bleeding as compared to opioids [odds ratios (OR): 1.30 and 1.02, respectively]. The risk of bleeding with ketorolac is strongly linked to increasing age, high dosages and treatment for more than 5 days. Ketorolac usually causes less nausea and vomiting than opioids. All NSAIDs have the potential to cause nephropathies, which, however, occur more frequently in patients with hypovolemia or other medical conditions that predispose them to hemodynamic compromise (D. J. Reinhart (2000) Drug Safety, Vol. 22, 487-497).

In order to reduce the occurrence of adverse effects related to the use of ketorolac, the maximum recommended intramuscular single-dose treatment of ketorolac in the US is 60 mg. The total daily dose is limited to 90 mg (UK, Italy, Sapin, Belgium, Switzerland) or 120 mg/day (US, Mexico, Canada, Finland and Sweden). The total therapeutic interval of ketorolac is recommended to be limited to 5 days (D. J. Reinhart (2000), supra).

In view of the aforesaid, the Applicant endeavored to prolong the duration of action of ketorolac. In this invention, several pharmaceutical compositions containing a ketorolac ester derivative and a selected oil vehicle were formulated. These compositions were demonstrated to exhibit long-acting analgesic and anti-inflammatory effects of several days, e.g., 3-5 days.

Specifically, in order to prepare a long-acting ketorolac preparation, a depot design with an esterification method, which is an established methodology for increasing the duration of a short-acting drug (K. S. Chu et al. (2003), supra), was used. This design involves the esterification of a drug to form a bioconvertible prodrug-type ester and the subsequent formulation of the prodrug-type ester in a pharmaceutically acceptable oil vehicle. The resultant oil preparation containing the prodrug-type ester is suitable for administration via intramuscular or subcutaneous injection and can form a drug reservoir at the site of injection. The rate of drug absorption is controlled by the interfacial partitioning of drug esters from the reservoir to the tissue fluid, and the rate of bioconversion of drug esters to regenerate active drug molecules.

With this design, this invention provides a ketorolac ester derivative of formula (I): 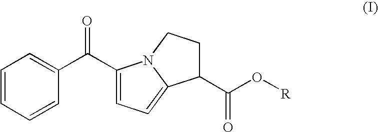
wherein
R is a straight-chain or branched saturated or unsaturated C₁-C₂₀ aliphatic group optionally substituted with a C₆-C₁₀ aryl group.

According to this invention, examples of the C₆-C₁₀ aryl group include phenyl, naphthyl, tetrahydronaphthyl, etc.

Preferably, R is a straight-chain or branched C₁-C₂₀ alkyl group optionally substituted with an aryl group. More preferably, R is a straight-chain or branched C₁-C₂₀ alkyl group. In a preferred embodiment of this invention, R is a straight-chain C₃-C₁₆ alkyl group, such as propyl, butyl, pentyl, hexyl, heptyl, decyl, cetyl, etc. In another preferred embodiment of this invention, R is a branched C₃-C₁₆ alkyl group, such as tert-butyl.

Preferably, R is a straight-chain or branched C₁-C₂₀ alkyl group substituted with an aryl group selected from phenyl, naphthyl and tetrahydronaphthyl. More preferably, R is a C₁-C₁₀ alkyl group substituted with a phenyl group. In a preferred embodiment of this invention, R is benzyl.

According to this invention, representatives of the ketorolac ester derivative of formula (I) are selected from the group consisting of ketorolac propyl ester, ketorolac t-butyl ester, ketorolac pentyl ester, ketorolac hexyl ester, ketorolac heptyl ester, ketorolac decyl ester, ketorolac cetyl ester, and ketorolac benzyl ester.

In a preferred embodiment of this invention, R is an aliphatic moiety derived from an aliphatic alcohol of formula ROH. The preferred ketorolac ester derivatives according to this invention can therefore be prepared from ketorolac and an alcohol selected from the group consisting of: propyl alcohol, tert-butyl alcohol, pentyl alcohol, hexyl alcohol, heptyl alcohol, benzyl alcohol, decyl alcohol, cetyl alcohol; saturated fatty alcohols, such as lauryl alcohol, stearyl alcohol, arachinyl alcohol, ceryl alcohol, etc.; and unsaturated fatty alcohol, such as oleyl alcohol, lanolin alcohol, undecylenyl alcohol, cinnamyl alcohol, etc.

The ketorolac ester derivative of formula (I) can be prepared by a process comprising:
(i) treating ketorolac tromethamine or base with 4-dimethylaminopyridine in the presence of tetrahydrofuran; and
(ii) adding to the resultant mixture of step (i) a compound of formula ROH in the presence of tetrahydrofuran, wherein R in the formula ROH is a straight-chain or branched saturated or unsaturated aliphatic group having 1 to 20 carbon atoms and optionally substituted with a C₆-C₁₀ aryl group; and
(iii) adding to the resultant mixture of step (ii) an appropriate coupling reagent, such as N,N′-dicyclohexylcarbodiimide (DCC), N,N′-carbonyldiimidazole, 1,1′-thionylimidazole and the like.

Preferably, a C₁-C₂₀ alkyl alcohol optionally substituted with a phenyl group, is used in step (ii).

Preferably, N,N′-dicyclohexylcarbodiimide (DCC) is used in the above step (iii) as the coupling reagent.

Specifically, to synthesize the ketorolac ester derivative of formula (I), ketorolac or ketorolac tromethamine was dissolved in tetrahydrofuran, followed by addition of 4-dimethylaminopyridine, which acted as a catalyst. Thereafter, the resultant mixture was added with a solution of a compound of formula ROH dropwise. Finally, a selected coupling reagent was added to the reaction mixture. Upon completion of esterfication, the reaction mixture was passed through a silical gel column, so that a ketorolac ester derivative of formula (I) was obtained.

As an alternative, the ketorolac ester derivatives of this invention may be obtained by the general method of preparing esters from alcohols or phenols, for instance, by reacting the carboxylic group of ketorolac with aliphatic alcohols, or various alcohols of formula ROH.

The ketorolac ester derivatives of formula (I) as synthesized by the methods described above can be identified by nuclear magnetic resonance (NMR), infrared (IR) and ultraviolet (UV) spectroscopy, and gas chromatography/mass spectrometry (GC/MS).

According to this invention, the synthesized ketorolac ester derivatives of formula (I) may be formulated into different pharmaceutical preparations so as to provide a long-acting therapeutic efficacy. In this regard, the ketorolac ester derivatives of formula (I) may be admixed with a selected oil vehicle to form a parenteral formulation, so that upon being administered to a subject such as a human or animal, the release rate of the target drug, i.e. ketorolac, may slow down due to the influence of some factors, e.g., the increased solubility of the target drug in oil. As a consequence, the dosing intervals of the target drug can be set longer by virtue of the prolonged duration of action thereof.

Gelders reported in International Clinical Psychopharmacology, (1986) Vol. 1, 1-11, and C. N. Hinko et al. reported in Neuropharmacology, (1988) Vol. 27, 475-483, the formation of a controlled-release dosage form of haloperidol decyl ester in an injectable oil, such as sesame oil or soybean oil, the antipsychotic effect of which was prolonged to make possible an extension of the dosing interval from 2 to 4 times a day to 1 to 2 times a month.

T. R. Norman reported in International Clinical Psychopharmacology, (1987) Vol. 2, 299-305, the preparation of fluphenazine decyl ester from fluphenazine. C. N. Hinko reported in Neuropharmacology, (1988), Vol. 27, 475-483, the preparation of an ester of nipectic acid. C. L. Broekkamp reported in Journal of Pharmacy and Pharmacology, (1988) Vol. 40, 434-437, the preparation of nicotinoyl morphine ester from morphine. J. V. Joshi et al. reported in Steroids, (1989) Vol. 53, 751-761, a precursor preparation of northisterone enenthate, which could be set with a longer dosing interval of up to two months.

However, due to unknown factors present in nature, quick release of a target drug from an oil vehicle could sometimes occur. For instance, the release of testosterone from the intramuscular administration of a testosterone suspension was found to be quick (T Tanaka (1974), Chemical & Pharmaceutical Bulletin, Vol. 22, pp. 1275-1284). H. A. C. Titulaer reported the addition of artemisinin in parenteral oil to form various dosage forms for intramuscular, intravenous, oral or rectal administration. However, the drug was released quickly from such dosage forms (Journal of Pharmacy and Pharmacology (1990), Vol. 42, pp. 810-813). Z. Zuidema et al. reported in International Journal of Pharmaceutics (1994), Vol. 105, pp. 189-207, that the release rate and extent of dosage forms for parenteral administration are very erratic and variable.

According to the aforementioned studies, a dosage form which contains a pharmaceutical composition suspended, or dissolved in an oil vehicle does not certainly exhibit a longer duration of therapeutic effect. In general, any attempt to add a target drug into an oil vehicle for the purpose of obtaining long-acting dosage forms need to take into account the physical solubility, stability, and release rate of the target drug from such vehicle.

In view of the aforesaid, in order to achieve the goal of extending the duration of action of ketorolac, the Applicant provided in this application an analgesic composition comprising a ketorolac ester derivative of formula (I) in admixture with a pharmaceutically acceptable oil vehicle.

The analgesic composition according to this invention is suitable for administration via intramuscular or subcutaneous route, and permits the controlled-release of the target drug—ketorolac—contained therein, thus providing a longer duration of action in relieving pain.

The oil vehicle suitable for use in this invention is injectable and includes, e.g., sesame oil, soybean oil, castor oil, cotton seed oil, peanut oil, and combinations thereof. Besides, the analgesic composition according to this invention may optionally comprise a pharmaceutically acceptable excipient that is commonly used in the manufacture of pharmaceuticals. The use of such an excipient will be readily apparent to persons skilled in the art. Preferably, the excipient, if present, may be selected from benzyl alcohol or chlorobutanol or combinations thereof.

The analgesic composition according to this invention has been proven to be able to provide a prolonged analgesia to a subject in need thereof. In addition, the analgesic composition according to this invention is also able to provide a prolonged anti-inflammatory effect to a subject in need thereof.

Accordingly, it is contemplated that this invention encompasses the use of a combination of a ketorolac ester derivative of formula (I) as described above and a pharmaceutically acceptable oil vehicle in the manufacture of an injectable long-acting analgesic composition that may provide a prolonged analgesia and anti-inflammatory effect to a subject in need thereof.

This invention also provides a method for providing a prolonged analgesia to a subject, comprising intramuscularly or subcutaneously administering an effective amount of the composition as described above to a subject in need of such treatment. Besides, this invention provides a method for providing a prolonged anti-inflammatory effect to a subject, comprising intramuscularly or subcutaneously administering an effective amount of the composition as described above to a subject in need of such treatment.

The long-acting analgesic composition of this invention can be administered once for several days. Even when the long-acting analgesic composition of this invention is administered with a larger dosage, the occurrence of undesired effects can be minimized.

The long-acting analgesic composition of this invention was found to have a prolonged duration of action, and such an advantage should improve therapeutic quality. The long-acting analgesic composition of this invention can therefore be set with a dosing interval of around 3 days instead of 6-8 hours for patients suffering from pain and inflammation.

This invention will be further described by way of the following examples. One of ordinary skill in the art is familiar with many techniques and teachings allowing the modification of these examples, and the examples noted throughout this disclosure that would also employ the basic, novel, or advantageous characteristics of the invention. Thus, the scope of this invention is not limited by the particular examples listed here or elsewhere.

### EXAMPLES

Table 1 shows the chemical structures of the preferred ketorolac ester derivatives obtained in the following Synthesis Examples.

TABLE 1
The chemical structures of ketorolac, ketorolac tromethamine, and
ketorolac ester derivatives obtained in the Synthesis Examples.

| Compound name | Chemical structure |
|---|---|
| Ketorolac | Keto—H |
| Ketorolac | Keto.Tromethamine |
| tromethamine | |
| Ketorolac propyl ester | 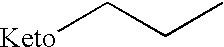 |
| Ketorolac pentyl ester | 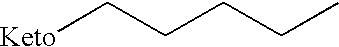 |
| Ketorolac tert-butyl ester | 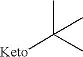 |
| Ketorolac hexyl ester | 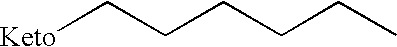 |
| Ketorolac heptyl ester | 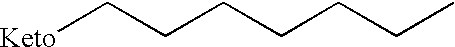 |
| Ketorolac decyl ester | 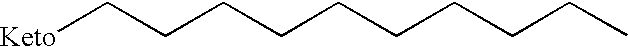 |
| Ketorolac cetyl ester | 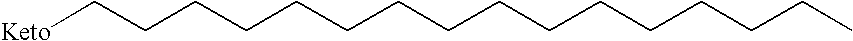 |
| Ketorolac benzyl ester | 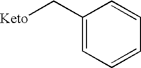 |

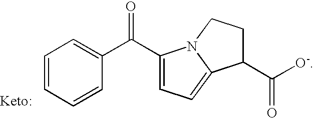

The ketorolac ester derivatives listed in Table 1 can be synthesized by suitable known methods other than those described below.

### Synthesis Ex. 1

### Preparation of Ketorolac Propyl Ester

Ketorolac tromethamine was purchased from Sigma (Saint Louis, Mo., USA). Ketorolac was obtained from its tromethamine salt using a precipitation method. Following adding 1 N HCl drop by drop into a ketorolac tromethamine solution, ketorolac was precipitated. The collected precipitate was purified by extraction with ethyl acetate, followed evaporation to dryness. The purity of ketorolac was checked by melting point measurement and HPLC analysis. The obtained product was measured to have a melting point of 155° C., virtually the same as reported in literature. The obtained product has a purity of >99% as determined by HPLC analysis.

To a 250-mL ice-bathed round-bottomed flask were added 45 mL of tetrahydrofuran (THF; Mallinckrodt Baker, New Jersy, USA) and 0.0135 mole of ketorolac. Subsequently, 0.0148 mole of propyl alcohol (Mallinckrodt Backer, New Jersey, USA) and 0.00135 mole of 4-dimethylaminopyridine (DMAP; Sigma, Missouri, USA) were gradually added into the flask with stirring. Finally, 0.0148 mole of N,N′-dicyclohexylcarbodiimide (DCC; Merck, Darmstadt, Germany) was added into the flask under argon gas.

Following stirring for 12 hrs, a waste product (N,N-dicyclohexylurea) was precipitated from the reaction mixture. After filtering off the precipitate, the remaining solution was concentrated by vacuum evaporation and then mixed with 100 ml of ethyl acetate. The resultant mixture was washed with 50 ml of 5% HCl and 5 ml of brine (saturated saline solution). The organic (ethyl acetate) layer was collected and concentrated by vacuum evaporation. The thus-obtained concentrate was subjected to column chromatography with 10% ethyl acetate in hexane, and purified ketorolac propyl ester was obtained.

The production of the title compound was affirmed by Tables 2-6, which summarized the physical characteristics, mass spectrum data, infra-red (IR) spectrum data, ultraviolet (UV) spectrum data and ¹H-NMR spectrum data of ketorolac propyl ester, respectively.

Detected Properties of the Title Compound:

Representative ¹H-NMR (400 MHz, CDCl₃): 7.82 (d,2H,Ar—H,J=7.9 Hz), 7.53 (m,1H,Ar—H), 7.45 (t,2H,Ar—H,J=7.7 Hz,7.4 Hz), 6.82 (d,1H,J=4.0 Hz), 6.11 (d,1H,J=4.1 Hz), 4.59-4.41 (m,2H), 4.16-4.05 (m,3H), 2.96-2.77 (m,2H), 1.74 (m,2H), 0.96 (t,3H,J=7.5 Hz,7.2 Hz).

Representative mass fragments (amu): 297, 210,105, 77 [detection was carried out using GC-MS spectroscopy (Spectrum RXI, Perkin Elmer, UK)]. Representative IR absorption (cm⁻¹): 2967.3, 1735.8, 1624.0, 1574.7, 1465.1, 1431.6, 1269.0 [detection was carried out using FT-IR spectroscopy (Spectrum RXI, Perkin Elmer, UK)].

### Synthesis Ex. 2

### Preparation of Ketorolac Tert-Butyl Ester

The title compound was prepared according to the procedures set forth in the above Synthesis Ex. 1, except that 0.0148 mole of tert-butyl alcohol (Kanto; Tokyo, Japan) was used in place of propyl alcohol. Purified ketorolac tert-butyl ester was obtained and affirmed by Tables 2 to 6, which summarized the physical characteristics, mass spectrum data, infra-red (IR) spectrum data, ultraviolet (UV) spectrum data and ¹H-NMR spectrum data of ketorolac tert-butyl ester, respectively.

Detected Properties of the Title Compound:

Representative ¹H-NMR (400 MHz, CDCl₃): 7.81 (d,2H,Ar—H,J=7.3 Hz); 7.52 (m,1H,Ar—H), 7.44 (t,2H,Ar—H,J=7.4 Hz,7.2 Hz), 6.81 (d,1H,J=3.9 Hz), 6.08 (d,1H,J=4.0 Hz), 4.60-4.37 (m,2H), 3.99-3.95 (m,1H), 2.93-2.69 (m,2H), 1.48 (s,9H).

Representative mass fragments (amu): 311, 255, 210, 105, 77 [detection was carried out using GC-MS spectroscopy (Spectrum RXI, Perkin Elmer, UK)]. Representative IR absorption (cm⁻¹): 2976.6, 1733.7, 1624.2, 1575.7, 1465.3, 1431.7, 1269.7 [detection was carried out using FT-IR spectroscopy (Spectrum RXI, Perkin Elmer, UK)].

### Synthesis Ex. 3

### Preparation of Ketorolac Pentyl Ester

The title compound was prepared according to the procedures set forth in the above Synthesis Ex. 1, except that 0.0148 mole of pentyl alcohol (Mallinckrodt Backer; New Jersy, USA) was used in place of propyl alcohol. Purified ketorolac pentyl ester was obtained and affirmed by Tables 2 to 6, which summarized the physical characteristics, mass spectrum data, infra-red (IR) spectrum data, ultraviolet (UV) spectrum data and ¹H-NMR spectrum data of ketorolac pentyl ester, respectively.

Detected properties of the title compound:

Representative ¹H-NMR (400 MHz, CDCl₃): 7.81 (d,2H,Ar—H,J=7.1 Hz), 7.54 (m,1H,Ar—H), 7.46 (t,2H,Ar—H,J=7.7 Hz,7.1 Hz), 6.82 (d,1H,J=3.9 Hz), 6.10 (d, 1H,J=3.8 Hz), 4.59-4.41 (m,2H), 4.17-4.13 (m,2H), 4.08-4.04 (m,1H), 2.96-2.77 (m,2H), 1.70-1.63 (m,2H), 1.36-1.32 (m,4H), 0.90 (t,3H,J=6.9 Hz,5.9 Hz).

Representative mass fragments (amu): 325, 210, 105, 77 [detection was carried out using GC-MS spectroscopy (Spectrum RXI, Perkin Elmer, UK)]. Representative IR absorption (cm⁻¹): 2956.6, 1736.0,1624.3, 1575.8, 1465.5, 1431.8, 1268.7 [detection was carried out using FT-IR spectroscopy (Spectrum RXI, Perkin Elmer, UK)].

### Synthesis Ex. 4

### Preparation of Ketorolac Hexyl Ester

The title compound was prepared according to the procedures set forth in the above Synthesis Ex. 1, except that 0.0148 mole of hexyl alcohol (Mallinckrodt Backer; New Jersy, USA) was used in place of propyl alcohol. Purified ketorolac hexyl ester was obtained and affirmed by Tables 2 to 6, which summarized the physical characteristics, mass spectrum data, infra-red (IR) spectrum data, ultraviolet (UV) spectrum data and ¹H-NMR spectrum data of ketorolac hexyl ester, respectively.

Detected Properties of the Title Compound:

Representative ¹H-NMR (400 MHz, CDCl₃): 7.81 (d,2H,Ar—H,J=7.4 Hz), 7.52 (m,1H,Ar—H), 7.44 (t,2H,Ar—H,J=7.7 Hz,7.4 Hz), 6.82 (d,1H,J=3.9 Hz), 6.09 (d,1H,J=4.2 Hz), 4.59-4.41 (m,2H), 4.17-4.13 (m,2H), 4.08-4.04 (m,1H), 2.96-2.77 (m,2H), 1.69-1.62 (m,2H), 1.39-1.30 (m,6H), 0.89 (t,3H,J=6.5 Hz,6.8 Hz).

Representative mass fragments (amu): 339, 210, 105, 77 [detection was carried out using GC-MS spectroscopy (Spectrum RXI, Perkin Elmer, UK)]. Representative IR absorption (cm⁻¹): 2930.6, 1731.8, 1621.2, 1575.6, 1463.3, 1433.3, 1268.7 [detection was carried out using FT-IR spectroscopy (Spectrum RXI, Perkin Elmer, UK)].

### Synthesis Ex. 5

### Preparation of Ketorolac Heptyl Ester

The title compound was prepared according to the procedures set forth in the above Synthesis Ex. 1, except that 0.0148 mole of heptyl alcohol (Mallinckrodt Backer; New Jersy, USA) was used in place of propyl alcohol. Purified ketorolac heptyl ester was obtained and affirmed by Tables 2 to 6, which summarized the physical characteristics, mass spectrum data, infra-red (IR) spectrum data, ultraviolet (UV) spectrum data and ¹H-NMR spectrum data of ketorolac heptyl ester, respectively.

Detected Properties of the Title Compound:

Representative ¹H-NMR (400 MHz, CDCl₃): 7.81 (d,2H,Ar—H,J=7.4 Hz), 7.52 (m, 1H,Ar—H), 7.44 (t,2H,Ar—H,J=7.4 Hz,7.6 Hz), 6.82 (d, 1H,J=3.9 Hz), 6.09 (d,1H,J=3.8 Hz), 4.59-4.41 (m,2H), 4.17-4.12 (m,2H), 4.08-4.04 (m,1H), 2.96-2.77 (m,2H), 1.69-1.62 (m,2H), 1.34-1.25 (m,8H), 0.88 (t,3H,J=6.2 Hz,7.0 Hz).

Representative mass fragments (amu): 353, 210,105, 77 [detection was carried out using GC-MS spectroscopy (Spectrum RXI, Perkin Elmer, UK)]. Representative IR absorption (cm⁻¹): 2928.7, 1737.6,1625.4, 1575.5, 1464.5, 1432.5, 1268.8 [detection was carried out using FT-IR spectroscopy (Spectrum RXI, Perkin Elmer, UK)].

### Synthesis Ex. 6

### Preparation of Ketorolac Decyl Ester

The title compound was prepared according to the procedures set forth in the above Synthesis Ex. 1, except that 0.0148 mole of decyl alcohol (Mallinckrodt Backer; New Jersy, USA) was used in place of propyl alcohol. Purified ketorolac decyl ester was obtained and affirmed by Tables 2 to 6, which summarized the physical characteristics, mass spectrum data, infra-red (IR) spectrum data, ultraviolet (UV) spectrum data and ¹H-NMR spectrum data of ketorolac decyl ester, respectively.

Detected properties of the title compound:

Representative ¹H-NMR (400 MHz, CDCl₃): 7.81 (d,2H,Ar—H,J=7.9 Hz), 7.52 (m,1H,Ar—H), 7.45 (t,2H,Ar—H,J=7.5 Hz,7.6 Hz), 6.82 (d,1H,J=3.9 Hz), 6.09 (d,1H,J=4.0 Hz), 4.59-4.41 (m,2H), 4.17-4.12 (m,2H), 4.08-4.04 (m,1H), 2.96-2.77 (m,2H), 1.66 (m,2H), 1.31-1.10 (m,14H), 0.87 (t,3H,J=6.4 Hz,6.9 Hz).

Representative mass fragments (amu): 395,290,210,105,77 [detection was carried out using GC-MS spectroscopy (Spectrum RXI, Perkin Elmer, UK)]. Representative IR absorption (cm⁻¹): 2925.8, 1736.1,1625.0, 1575.9, 1465.6, 1431.9, 1268.8 [detection was carried out using FT-IR spectroscopy (Spectrum RXI, Perkin Elmer, UK)].

### Synthesis Ex. 7

### Preparation of Ketorolac Cetyl Ester

The title compound was prepared according to the procedures set forth in the above Synthesis Ex. 1, except that 0.0148 mole of cetyl alcohol (Mallinckrodt Backer; New Jersy, USA) was used in place of propyl alcohol. Purified ketorolac cetyl ester was obtained and affirmed by Tables 2 to 6, which summarized the physical characteristics, mass spectrum data, infra-red (IR) spectrum data, ultraviolet (UV) spectrum data and ¹H-NMR spectrum data of ketorolac cetyl ester, respectively.

Detected properties of the title compound:

Representative ¹H-NMR (400 MHz, CDCl₃): 7.81 (d,2H,Ar—H,J=7.0 Hz), 7.52 (m,1H,Ar—H), 7.45 (t,2H,Ar—H,J=6.5 Hz,6.9 Hz), 6.82 (d,1H,J=3.9 Hz), 6.09 (d,1H,J=4.3 Hz), 4.59-4.43 (m,2H), 4.17-4.04 (m,3H), 2.96-2.77 (m,2H), 1.69-1.62 (m,2H), 1.31-1.25 (m,26H), 0.88 (t,3H,J=6.5 Hz,7.0 Hz).

Representative mass fragments (amu): 479, 374, 210, 105 [detection was carried out using GC-MS spectroscopy (Spectrum RXI, Perkin Elmer, UK)]. Representative IR absorption (cm⁻¹): 2923.2, 1738.1, 1626.0, 1575.8, 1463.7, 1433.2, 1268.6 [detection was carried out using FT-IR spectroscopy (Spectrum RXI, Perkin Elmer, UK)].

### Synthesis Ex. 8

### Preparation of Ketorolac Benzyl Ester

The title compound was prepared according to the procedures set forth in the above Synthesis Ex. 1, except that 0.0148 mole of benzyl alcohol (Mallinckrodt Backer; New Jersy, USA) was used in place of propyl alcohol. Pure ketorolac benzyl ester was obtained and affirmed by Tables 2 to 6, which summarized the physical characteristics, mass spectrum data, infra-red (IR) spectrum data, ultraviolet (UV) spectrum data and ¹H-NMR spectrum data of ketorolac benzyl ester, respectively.

Detected properties of the title compound:

Representative ¹H-NMR (400 MHz, CDCl₃): 7.81 (d,2H,Ar—H,J=7.4 Hz), 7.52 (m,1H,Ar—H), 7.44 (t,2H,Ar—H,J=7.7 Hz,7.2 Hz), 7.37-7.32 (m,5H,Ar—H), 6.80 (d,1H,J=4.0 Hz), 6.07 (d,1H,J=3.8 Hz), 5.19 (s,2H), 4.61-4.41 (m,2H), 4.13-4.09 (m,1H), 2.97-2.76 (m,2H).

Representative mass fragments (amu): 345, 210, 105, 91, 77 [detection was carried out using GC-MS spectroscopy (Spectrum RXI, Perkin Elmer, UK)]. Representative IR absorption (cm⁻¹): 2955.9, 1736.2,1623.6, 1574.3, 1464.9, 1431.3, 1268.7 [detection was carried out using FT-IR spectroscopy (Spectrum RXI, Perkin Elmer, UK)].

TABLE 2
The physical characteristics of ketorolac, ketorolac
tromethamine and eight synthesized ketorolac esters

| | | | | Ester |
|---|---|---|---|---|
| Compound | | | | linkage |
| name | MW | MF | MP (° C.) | IR (cm⁻¹) |
| Ketorolac | 255.3 | C₁₅H₁₃NO₃ | 154˜156 | — |
| ketorolac | 376.4 | C₁₅H₁₂NO₃.C₄H₁₂NO₃ | 159˜161 | — |
| tromethamine | | | | |
| Ketorolac | 297.4 | C₁₈H₁₉NO₃ | <0 | 1735.80 |
| propyl ester | | | | |
| Ketorolac | 311.4 | C₁₉H₂₁NO₃ | 96˜98 | 1733.74 |
| tert-butyl ester | | | | |
| Ketorolac | 325.4 | C₂₀H₂₃NO₃ | <0 | 1736.02 |
| pentyl ester | | | | |
| Ketorolac | 339.4 | C₂₁H₂₅NO₃ | <0 | 1731.83 |
| hexyl ester | | | | |
| Ketorolac | 353.5 | C₂₂H₂₇NO₃ | 38˜40 | 1737.60 |
| heptyl ester | | | | |
| Ketorolac | 395.6 | C₂₅H₃₃NO₃ | 23˜25 | 1736.10 |
| decyl ester | | | | |
| Ketorolac | 479.7 | C₃₁H₄₅NO₃ | 45˜47 | 1738.17 |
| cetyl ester | | | | |
| Ketorolac | 345.4 | C₂₂H₁₉NO₃ | <0 | 1736.25 |
| benzyl ester | | | | |

Infra-red spectrum of each compound was detected using FT-IR spectroscopy (Spectrum RXI, Perkin Elmer, UK)

TABLE 3
The mass spectrum data of eight synthesized
ketorolac ester derivatives

| Compound name | mass fragments (amu) |
|---|---|
| Ketorolac propyl ester | 297, 210, 105, 77 |
| Ketorolac tert-butyl ester | 311, 255, 210, 105, 77 |
| Ketorolac pentyl ester | 325, 210, 105, 77 |
| Ketorolac hexyl ester | 339, 210, 105, 77 |
| Ketorolac heptyl ester | 353, 210, 105, 77 |
| Ketorolac decyl ester | 395, 290, 210, 105, 77 |
| Ketorolac cetyl ester | 479, 374, 210, 105 |
| Ketorolac benzyl ester | 345, 210, 105, 91, 77 |

Detected using GC-MS spectroscopy (Spectrum RXI, Perkin Elmer, UK)

TABLE 4
The infra-red spectrum data of eight synthesized
ketorolac ester derivatives

| Compound name | IR absorption (cm⁻¹) |
|---|---|
| Ketorolac propyl ester | 2967.3, 1735.8, 1624.0, 1574.7, |
| | 1465.1, 1431.6, 1269.0 |
| Ketorolac tert-butyl ester | 2976.6, 1733.7, 1624.2, 1575.7, |
| | 1465.3, 1431.7, 1269.7 |
| Ketorolac pentyl ester | 2956.6, 1736.0, 1624.3, 1575.8, |
| | 1465.5, 1431.8, 1268.7 |
| Ketorolac hexyl ester | 2930.6, 1731.8, 1621.2, 1575.6, |
| | 1463.3, 1433.3, 1268.7 |
| Ketorolac heptyl ester | 2928.7, 1737.6, 1625.4, 1575.5, |
| | 1464.5, 1432.5, 1268.8 |
| Ketorolac decyl ester | 2925.8, 1736.1, 1625.0, 1575.9, |
| | 1465.6, 1431.9, 1268.8 |
| Ketorolac cetyl ester | 2923.2, 1738.1, 1626.0, 1575.8, |
| | 1463.7, 1433.2, 1268.6 |
| Ketorolac benzyl ester | 2955.9, 1736.2, 1623.6, 1574.3, |
| | 1464.9, 1431.3, 1268.7 |

Detected using FT-IR spectroscopy (Spectrum RXI, Perkin Elmer, UK)

TABLE 5
The ultraviolet spectrum data of eight
synthesized ketorolac ester derivatives

| Compound name | Absorption (nm) |
|---|---|
| Ketorolac propyl ester | 245, 311 |
| Ketorolac tert-butyl ester | 245, 312 |
| Ketorolac pentyl ester | 245, 312 |
| Ketorolac hexyl ester | 245, 311 |
| Ketorolac heptyl ester | 243, 312 |
| Ketorolac decyl ester | 244, 312 |
| Ketorolac cetyl ester | 245, 311 |
| Ketorolac benzyl ester | 246, 311 |

Detected using ultraviolet spectroscopy (Spectrum RXI, Perkin Elmer, UK)

TABLE 6
The proton nuclear magnetic resonance spectrum data
of eight synthesized ketorolac ester derivatives

| Compound name | ¹H-NMR(400 MHz, CDCl₃) |
|---|---|
| Ketorolac propyl ester | 7.82(d, 2H, Ar—H, J=7.9Hz), 7.53(m, 1H, Ar—H), |
| | 7.45(t, 2H, Ar—H, J=7.7Hz, 7.4Hz), 6.82(d, 1H, J=4.0Hz), |
| | 6.11(d, 1H, J=4.1Hz), 4.59−4.41(m, 2H), 4.16−4.05(m, 3H), |
| | 2.96−2.77(m, 2H), 1.74(m, 2H), 0.96(t, 3H, J=7.5Hz, 7.2Hz) |
| Ketorolac tert-butyl ester | 7.81(d, 2H, Ar—H, J=7.3Hz), 7.52(m, 1H, Ar—H), |
| | 7.44(t, 2H, Ar—H, J=7.4Hz, 7.2Hz), 6.81(d, 1H, J=3.9Hz) |
| | 6.08(d, 1H, J=4.0Hz), 4.60−4.37(m, 2H), 3.99−3.95(m, 1H), |
| | 2.93−2.69(m, 2H), 1.48(s, 9H) |
| Ketorolac pentyl ester | 7.81(d, 2H, Ar—H, J=7.1Hz), 7.54(m, 1H, Ar—H), |
| | 7.46(t, 2H, Ar—H, J=7.7Hz, 7.1Hz), 6.82(d, 1H, J=3.9Hz) |
| | 6.10(d, 1H, J=3.8Hz), 4.59−4.41(m, 2H), 4.17−4.13(m, 2H), |
| | 4.08−4.04(m, 1H), 2.96−2.77(m, 2H), 1.70−1.63(m, 2H), |
| | 1.36−1.32(m, 4H), 0.90(t, 3H, J=6.9Hz, 5.9Hz) |
| Ketorolac hexyl ester | 7.81(d, 2H, Ar—H, J=7.4Hz), 7.52(m, 1H, Ar—H), |
| | 7.44(t, 2H, Ar—H, J=7.7Hz, 7.4Hz), 6.82(d, 1H, J=3.9Hz) |
| | 6.09(d, 1H, J=4.2Hz), 4.59−4.41(m, 2H), 4.17−4.13(m, 2H), |
| | 4.08−4.04(m, 1H), 2.96−2.77(m, 2H), 1.69−1.62(m, 2H), |
| | 1.39−1.30(m, 6H), 0.89(t, 3H, J=6.5Hz, 6.8Hz) |
| Ketorolac heptyl ester | 7.81(d, 2H, Ar—H, J=7.4Hz), 7.52(m, 1H, Ar—H), |
| | 7.44(t, 2H, Ar—H, J=7.4Hz, 7.6Hz), 6.82(d, 1H, J=3.9Hz), |
| | 6.09(d, 1H, J=3.8Hz), 4.59−4.41(m, 2H), 4.17−4.12(m, 2H), |
| | 4.08−4.04(m, 1H), 2.96−2.77(m, 2H), 1.69−1.62(m, 2H), |
| | 1.34−1.25(m, 8H), 0.88(t, 3H, J=6.2Hz, 7.0Hz) |
| Ketorolac decyl ester | 7.81(d, 2H, Ar—H, J=7.9Hz), 7.52(m, 1H, Ar—H), |
| | 7.45(t, 2H, Ar—H, J=7.5Hz, 7.6Hz), 6.82(d, 1H, J=3.9Hz), |
| | 6.09(d, 1H, J=4.0Hz), 4.59−4.41(m, 2H), 4.17−4.12(m, 2H), |
| | 4.08−4.04(m, 1H), 2.96−2.77(m, 2H), 1.66(m, 2H), |
| | 1.31−1.10(m, 14H), 0.87(t, 3H, J=6.4Hz, 6.9Hz) |
| Ketorolac cetyl ester | 7.81(d, 2H, Ar—H, J=7.0Hz), 7.52(m, 1H, Ar—H), |
| | 7.45(t, 2H, Ar—H, J=6.5Hz, 6.9Hz), 6.82(d, 1H, J=3.9Hz), |
| | 6.09(d, 1H, J=4.3Hz), 4.59−4.43(m, 2H), 4.17−4.04(m, 3H), |
| | 2.96−2.77(m, 2H), 1.69−1.62(m, 2H), 1.31−1.25(m, 26H), |
| | 0.88(t, 3H, J=6.5Hz, 7.0Hz) |
| Ketorolac benzyl ester | 7.81(d, 2H, Ar—H, J=7.4Hz), 7.52(m, 1H, Ar—H), |
| | 7.44(t, 2H, Ar—H, J=7.7Hz, 7.2Hz), 7.37−7.32(m, 5H, Ar—H), |
| | 6.80(d, 1H, J=4.0Hz), 6.07(d, 1H, J=3.8Hz), 5.19(s, 2H), |
| | 4.61−4.41(m, 2H), 4.13−4.09(m, 1H), 2.97−2.76(m, 2H) |

### Preparation Ex. 1

### Preparation of Injectable Long-Acting Analgesic Compositions

800 μmole of a ketorolac ester derivative of formula (I), such as any one of those synthesized in Synthesis Examples 1-8, may be admixed with 1 mL of an injectable oil vehicle selected from sesame oil, soybean oil, castor oil, cotton seed oil, peanut oil, or combinations thereof. The resultant mixture is then shaken slightly to effect complete dissolution. Representatives of the injectable long-acting analgesic compositions of this invention are given below.
(1) 800 μmole of ketorolac propyl ester was admixed with 1 mL of injectable sesame oil. The resultant mixture was shaken slightly to effect complete dissolution.
(2) 800 μmole of ketorolac heptyl ester was admixed with 1 mL of injectable sesame oil. The resultant mixture was shaken slightly to effect complete dissolution.
(3) 800 μmole of ketorolac cetyl ester was admixed with 1 mL of injectable sesame oil. The resultant mixture was shaken slightly to effect complete dissolution.

### Pharmacological Ex. 1

### Evaluation of the In Vivo Analgesic Efficacy and Anti-Inflammatory Effect of Intramuscularly Administered Ketorolac Tromethamine in Rats Intraplantarly Injected with Carrageenin (Dose-Finding Study)

(1) Animal: male Sprague-Dawley rats (175-225 gm, 6 weeks old), n=6 in each group of different doses.
(2) Study design: All rats received one intramuscular injection of either ketorolac tromethamine or vehicle (0.9% saline) at the start of the study and then (1 minute later) received intraplantar injection of carrageenin. Subsequent to carrageenin injection, rats were observed for a period of 10 hours to determine the analgesic and anti-inflammatory effects of ketorolac tromethamine.
(3) Analgesic drugs: ketorolac tromethamine, prepared in 0.9% saline (solution), doses in use: 8 μmole/Kg (=3 mg/Kg), 24 μmole/Kg (=9 mg/Kg), 80 μmole/Kg (=30 mg/Kg), 240 μmole/Kg (=90 mg/Kg). Each dose was intramuscularly injected in the right hind leg of rats in a volume of 0.1 ml.
(4) Carrageenin injection (a model of inflammation): The intraplantar injection of carrageenin has been widely used to produce a model of localized inflammatory pain (D. Fletcher et al. (1997), Anesthesia Analgesia, Vol. 84, 90-94). One minute after ketorolac tromethamine injection, 100 μL 1% λ-carrageenin (Sigma-Aldrich, St. Louis, Mo., USA) was injected subcutaneously into the plantar space of the right hind paw of all rats. Intraplantar injections were made with a Hamilton syringe and a 30-gauge hypodermic needle. The needle was inserted into the pad region of the glabrous skin and moved 6-8 mm proximal toward the tarsal region.
(5) Measurements of the pain threshold values of rat paw: Paw pressure test was conducted using the TSE Analgesia System (TSE Technical & Scientific Equipment GmbH; Bad Homburg, Germany).

The TSE Analgesia System is designed to perform rapid and accurate screening of analgesic drug candidates on the normal and inflamed paw of small laboratory animals, according to the Randall-Selitto method. The data can be measured and recorded by a control unit and a computer system connected thereto. The animal's paw is placed on a plinth and is applied with an increasing pressure generated from the tip of a sensor. The applied pressure is measured. The sensor is made from smooth plastic to prevent the paw from being injured when the animal withdraws its paw suddenly.

To start the test, a foot switch is pressed and the sensor is lowered rapidly until it contacts the rat's paw. Thereafter, the sensor is lowered in small steps until the pain threshold is reached, at which time the tested animal (rat) starts to move vigorously and tries to withdraw its paw. The foot switch is then released and the sensor is raised. The value of the applied pressure measured at the pain threshold is shown on the display and simultaneously delivered to the computer system, which is installed with a software system capable of transferring the measured data into Excel format, which can be used as a basis for further evaluation, e.g., statistics. The TSE Analgesia system is suitable for rats, mice and other small laboratory animals.

The pressure baseline of paw withdrawal in rats of the study was around 140-190 gm. To prevent tissue damage, a cutoff pressure of 350 gm was set.

For further details of the paw pressure test, reference may be made to, e.g., T. Pelissier et al. (2001), European Journal of Pharmacology, Vol. 416, 51-57, and D. Fletcher et al. (1997), Anesthesia Analgesia, Vol. 84, 90-94.
(6) Measurement of paw swelling: The anti-inflammatory effect of ketorolac tromethamine on paw swelling following intraplantar injection of carrageenin was assessed by changes in paw thickness (cm) using JOCAL calipers. Paw thickness was measured immediately prior to and following carrageenin injection.

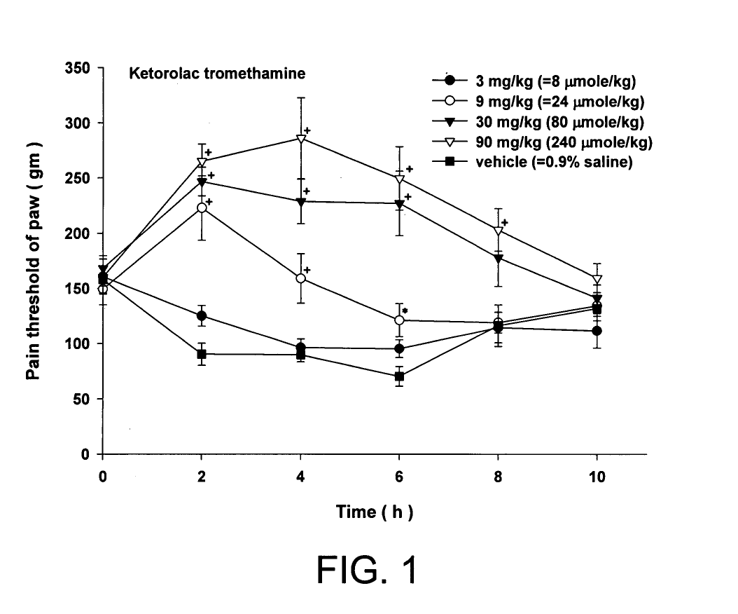

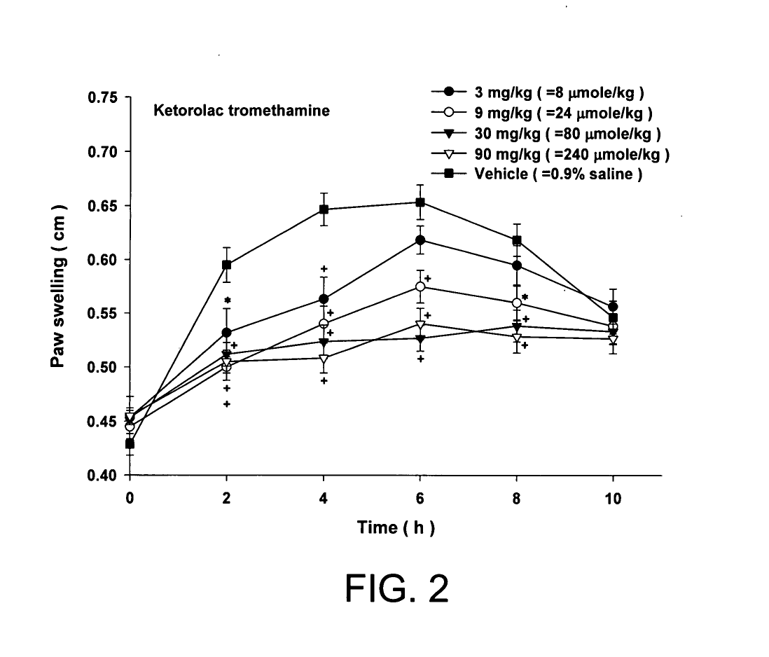

As for details of the intraplantar injection of carrageenin, reference may be made to, e.g., D. Fletcher et al. (1996), Anesthesiology, Vol. 84, 1129-1137, and M. J. Sammons et al. (2000), Brain Research, Vol. 876, 48-54.
(7) Statistics: Data are shown as mean±standard error. A two-way analysis of variance with one-way repeated method was used to compare the differences between groups. The Bonferroni test was used as a post-hoc test to compare the differences between the medication groups and the vehicle group at each time point. A P value less than 0.05 was considered significant. (“*” means P<0.05 whereas “+” means P<0.01 when compared with the vehicle group). The two-way analysis of variance with one-way repeated method is a powerful statistical method which can be used to evaluate the differences among groups. The Bonferroni test is a statistical method which can be used to compare the differences between groups.
(8) Results: Ketorolac tromethamine produced dose-related analgesic and anti-inflammatory effects upon rats (see Table 7 and FIGS. 1-2). Intramuscular injection of 24-240 μmole ketorolac tromethamine produced significant analgesic effects (as determined by the results of pain threshold of paw) of 6-8 hours and significant anti-inflammatory effects (as determined by the results of paw swelling) of 8 hours. A 10-fold increase (24 to 240) in the administered dose did not give a great improvement in the duration of action of ketorolac tromethamine.

### Pharmacological Ex. 2

### In Vivo Dose-Finding Studies of Intramuscularly Administered Ketorolac Propyl Ester (Prepared in Sesame Oil) in Rats Intraplantarly Injected with Carrageenin

(1) Animal: male Sprague-Dawley rats (175-225 gm, 6 weeks old), n=6 in each group of different doses at each day of study.
(2) Study design: A 4-day study was carried out. Subsequent to intraplantar injection of carrageenin, paw edema and pain occurred gradually with a maximum intensity at 6 hours and then gradually reduced (D. Fletcher et al. (1997), Anesthesia Analgesia, 84, 90-94).

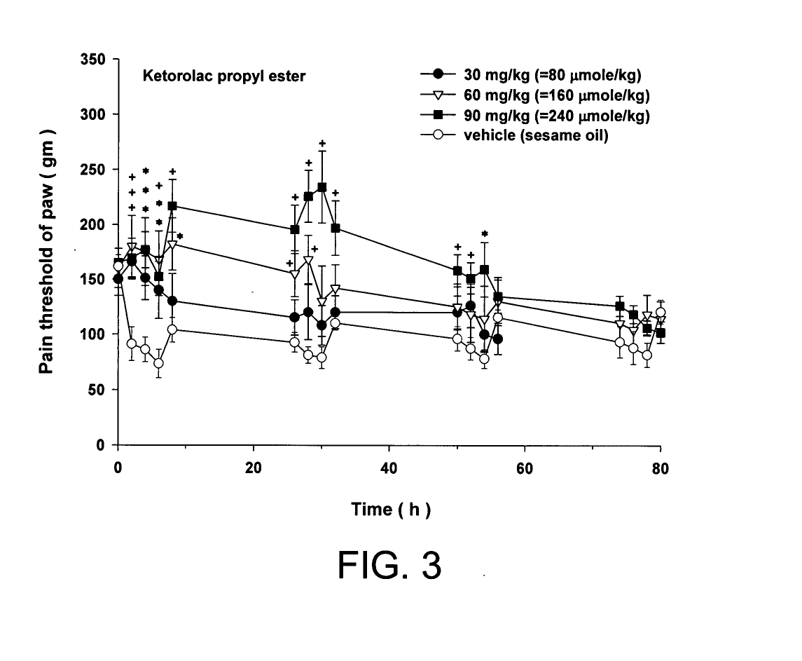

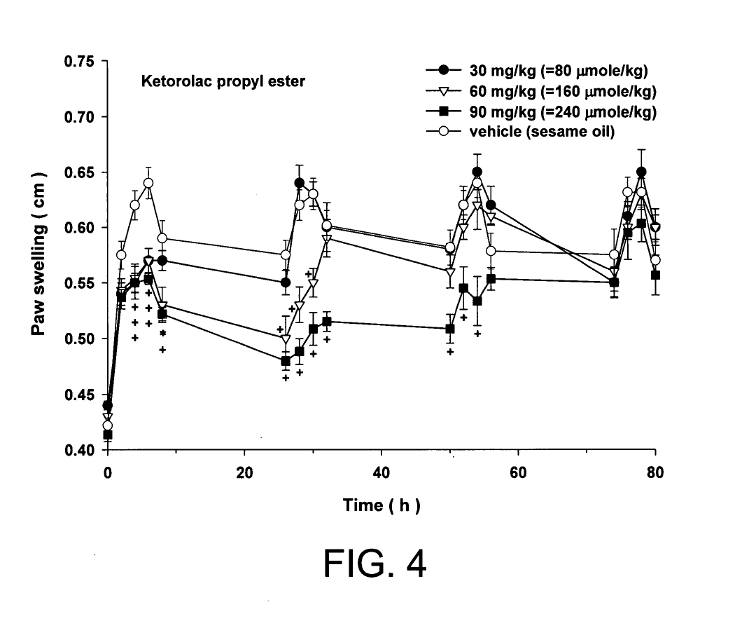

In order to keep a similar condition of carrageenin-induced paw edema and pain at each testing day, this 4-day study was accomplished by conducting four one-day studies consecutively (i.e. from day 1 to day 4). All rats received only one intramuscular injection of either ketorolac propyl ester (80, 160, or 240 μmole/Kg, prepared in sesame oil) or the vehicle (sesame oil only) at the start of study (day 1) and then (1 minute later) received intraplantar injection of carrageenin at either day 1, 2, 3, or day 4. Each rat received only one injection of carrageenin. Subsequent to carrageenin injection, rats were observed for a period of 6 hours to determine the analgesic and anti-inflammatory effects of ketorolac propyl ester.
(3) Analgesic drugs: ketorolac propyl ester, prepared in sesame oil, doses in use: 80 μmole/Kg, 160 μmole/Kg, 240 μmole/Kg. Each dose was intramuscularly injected in the right hind leg of rats in a volume of 0.1 ml.
(4) Carrageenin injection: See Pharmacological Example 1 described above.
(5) Measurements of the pain threshold values of rat paw: See Pharmacological Example 1 described above.
(6) Measurement of paw swelling: See Pharmacological Example 1 described above.
(7) Statistics: Data are shown as mean±standard error. A three-way analysis of variance with one-way repeated method was used to compare the differences between groups. The Bonferroni test was used as a post-hoc test to compare the differences between the medication groups and the vehicle group at each time point. A P value less than 0.05 was considered significant. (“*” means P<0.05 whereas “+” means P<0.01 when compared with the vehicle group). The three-way analysis of variance with one-way repeated method is a powerful statistical method which can be used to evaluate the differences among groups. The Bonferroni test is a statistical method which can be used to compare the differences between groups.
(8) Results: Intramuscular injection of different doses of ketorolac propyl ester prepared in sesame oil produced long durations of analgesic action and anti-inflammatory effects (see Table 7 and FIGS. 3-4).

### Pharmacological Ex. 3

### Evaluation of the In Vivo Analgesic Efficacy and Anti-Inflammatory Effect of Intramuscularly Administered Ketorolac and Six Ketorolac Ester Derivatives of Formula (I) (Prepared in Sesame Oil) in Rats Intraplantarly Injected with Carrageenin

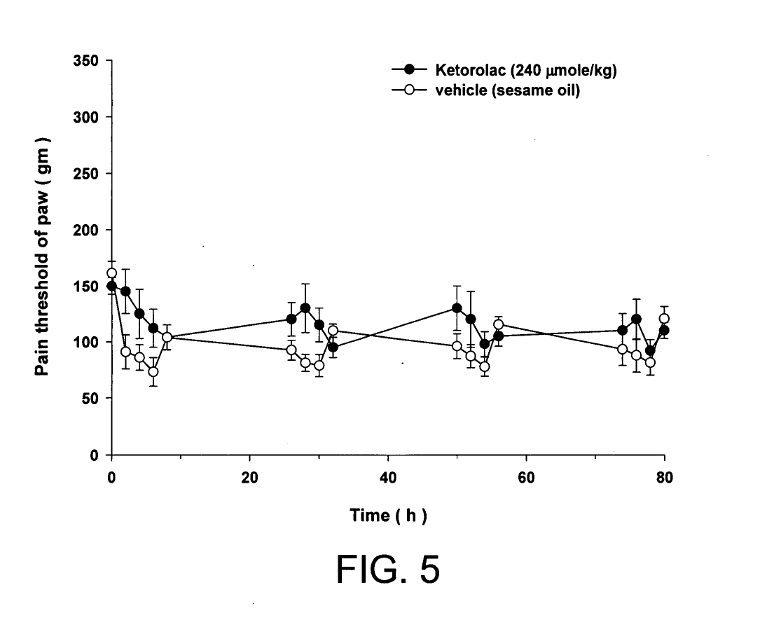

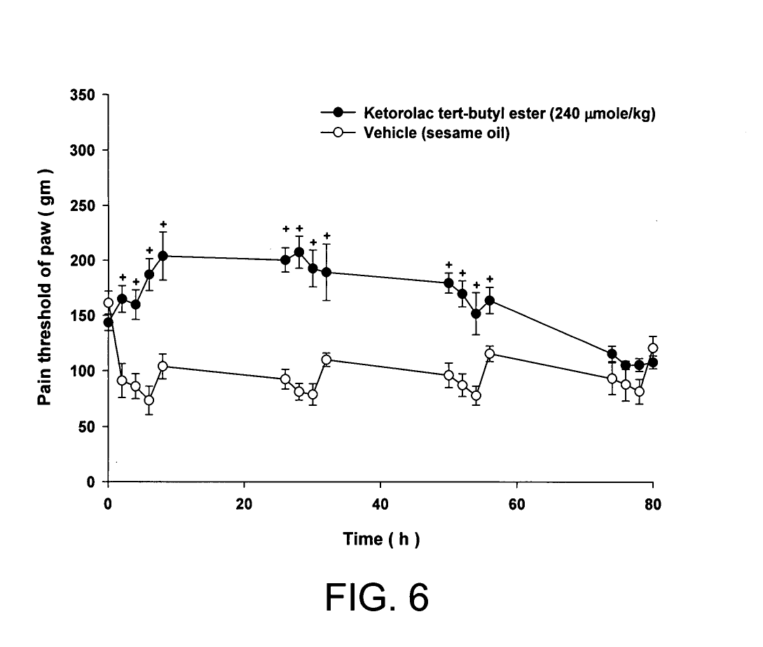

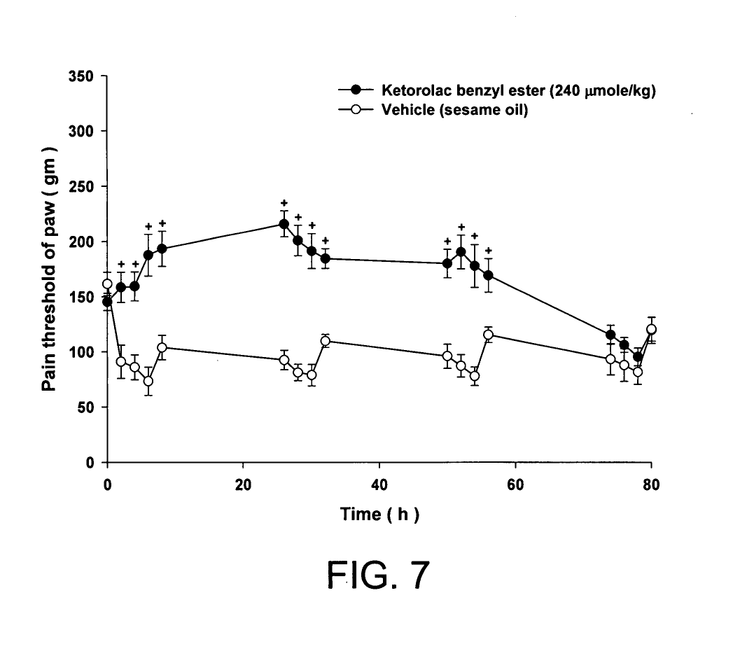

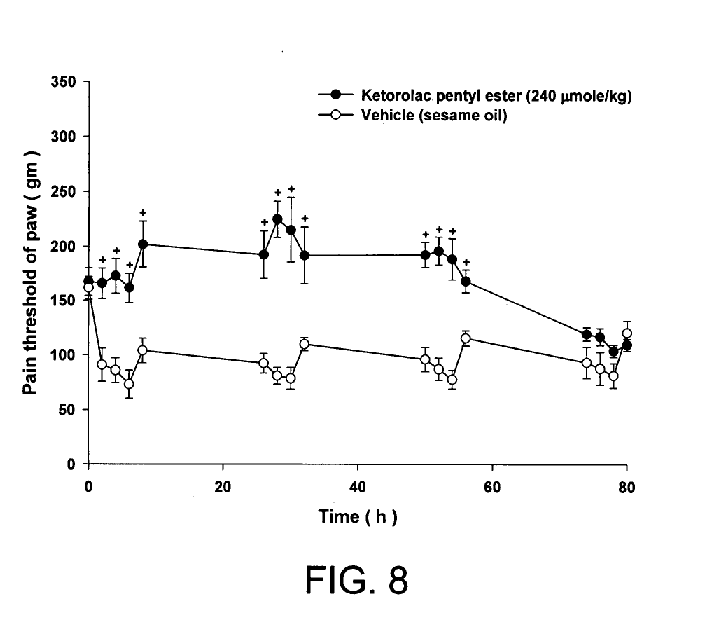

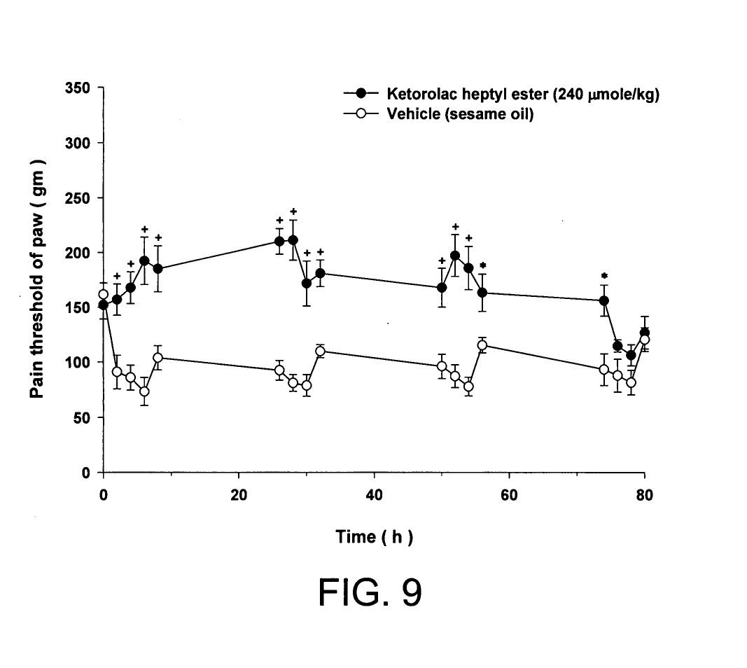

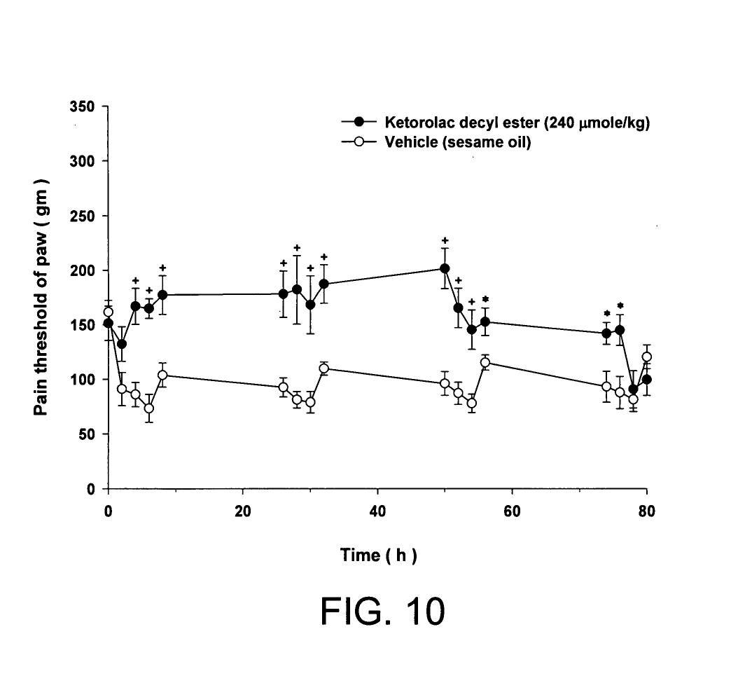

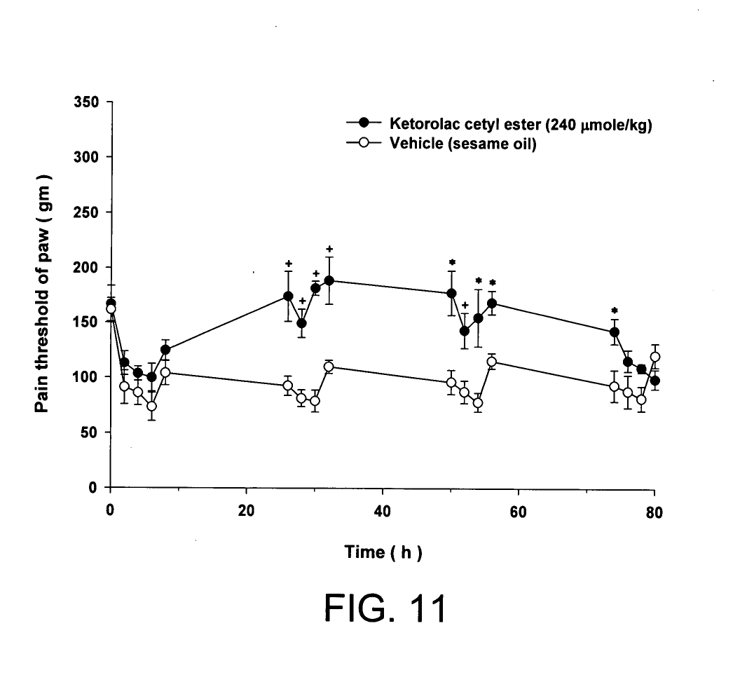

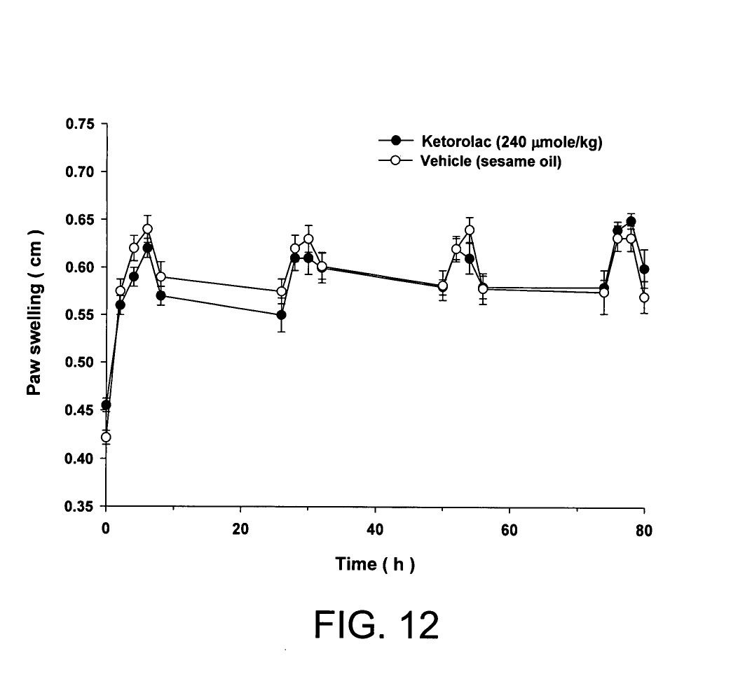

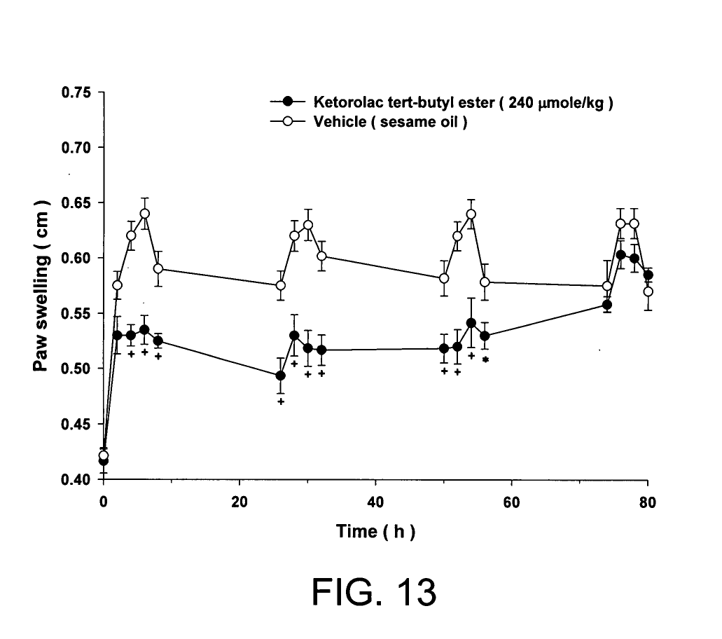

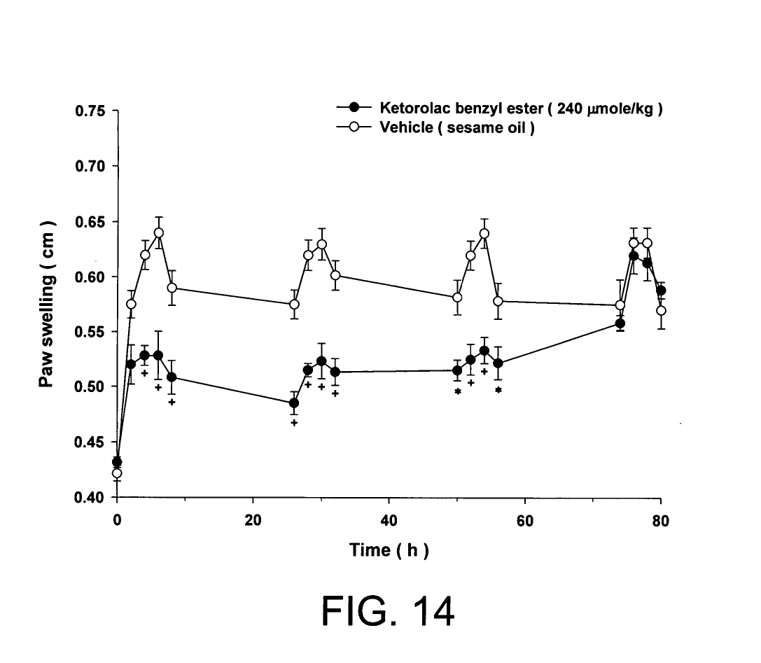

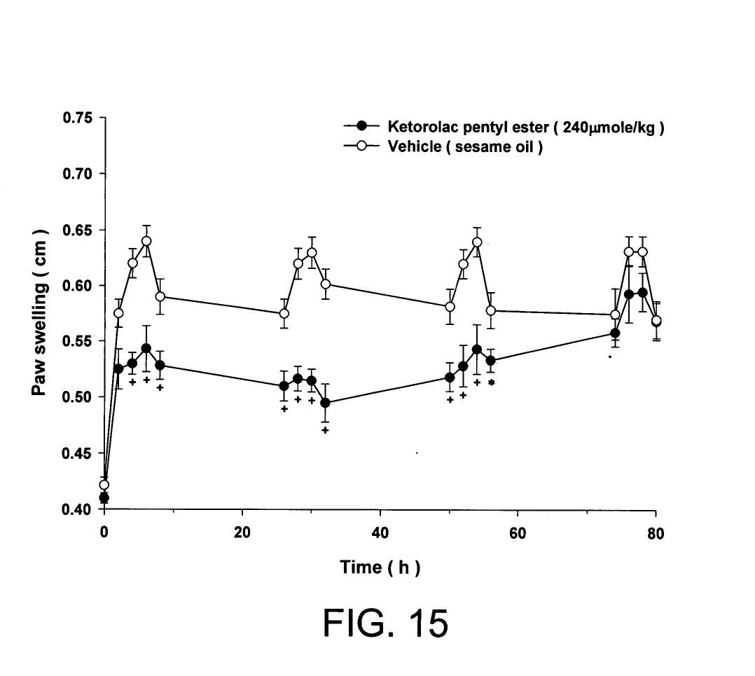

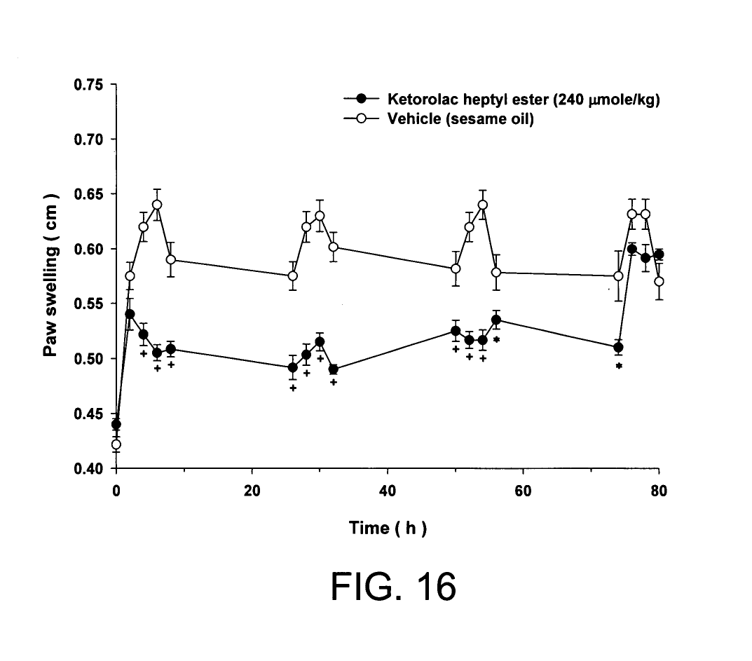

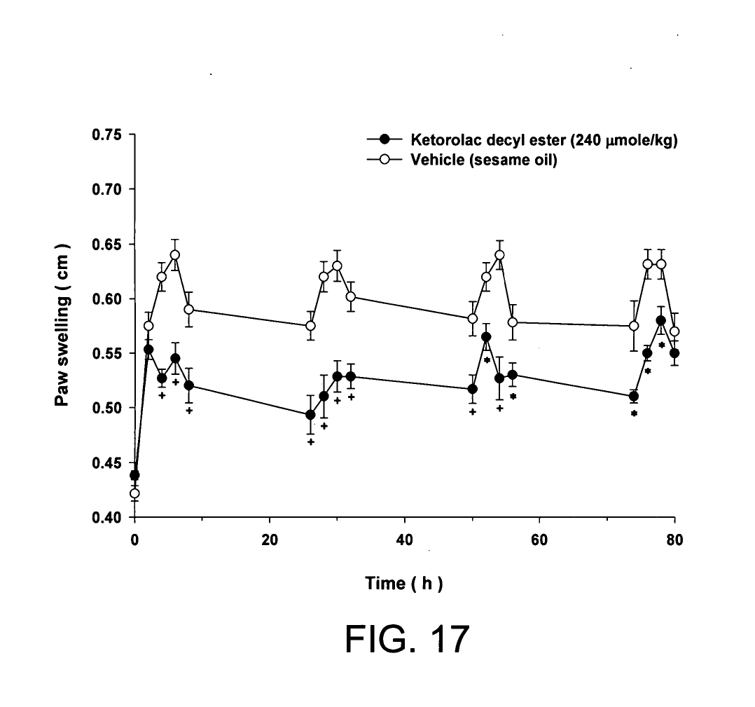

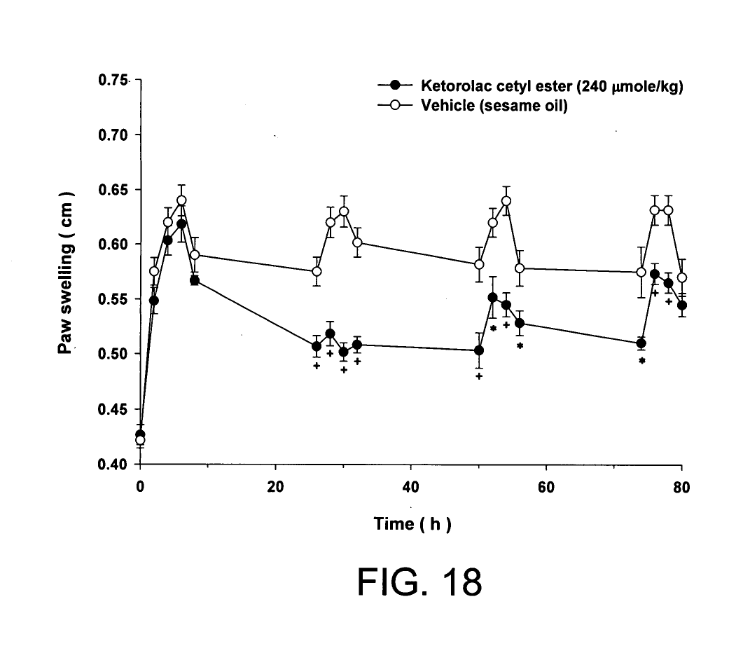

(1) Animal: male Sprague-Dawley rats (175-225 gm, 6 weeks old), n=6 in each group of each analgesic drug at each day of study.
(2) Study design: A 4-day study was carried out. This 4-day study was accomplished by conducting four one-day studies consecutively (see Pharmacological Example 2 described above). All rats received only one intramuscular injection of a tested analgesic drug (ketorolac or a ketorolac ester derivative, prepared in sesame oil) or the vehicle (sesame oil only) at the start of study (day 1) and then received intraplantar injection of carrageenin at either day 1, 2, 3, or day 4. Each rat received only one injection of carrageenin. Subsequent to carrageenin injection, rats were observed for a period of 8 hours to determine the analgesic and anti-inflammatory effects of the tested analgesic drugs.
(3) Analgesic drugs: ketorolac and six ketorolac ester derivatives (tert-butyl ester, benzyl ester, pentyl ester, heptyl ester, decyl ester, and cetyl ester), all prepared in sesame oil, dose in use: 240 μmole/Kg. All the ester derivatives were dissolved in sesame oil as an oil solution, whereas Ketorolac was prepared in sesame oil as an oil suspension (the solubility of ketorolac in sesame oil was low, 2.7 mg/mL=10.6 mmole/mL). Each analgesic drug was intramuscularly injected in the right hind leg of rats in a volume of 0.1 ml.
(4) Carrageenin injection: See Pharmacological Example 1 described above.
(5) Measurements of the pain threshold values of rat paw: See Pharmacological Example 1 described above.
(6) Measurement of paw swelling: See Pharmacological Example 1 described above.
(7) Statistics: The three-way analysis of variance with one-way repeated method followed by the Bonferroni test was used (see Pharmacological Example 2 described above).
(8) Results: Ketorolac prepared in sesame oil failed to provide significant analgesic and antiinflamatory effects due to the limited solubility of ketorolac in sesame oil (see FIGS. 5 and 12). In contrast, intramuscular injection of six ketorolac ester derivatives of formula (I) prepared in sesame oil at a dose of 240 μmole/Kg produced long durations of analgesic actions and anti-inflammatory effects (see Table 7, FIGS. 5-11, and FIGS. 12-18). The duration of actions of these ester derivatives were around 54-78 hours.

In clinical practice, ketorolac tromethamine 30 mg (=80 μmole) given to an adult provides a 6- to 8-hour duration of action. In this invention, intramuscular injection of ketorolac tromethamine 30 mg/kg in rats provided a 6- to 8-hour duration of action. According to the ratio (1) obtained from humans and rats (6-8 hrs/6-8 hrs), it is estimated that intramuscular injection of ketorolac ester derivatives at a proper dose, e.g., 240 mmole, may provide a duration of action lasting for around 54 to 78 hours. Since most patients who have acute pain, such as post-operative pain, traumatic pain, and burn pain, oftentimes require analgesics in the first three days after injury (K. S. Chu, et al. (2003), supra), intramuscular injection of a ketorolac ester derivative of formula (I) may be a suitable alternative for the management of pain in these patients.

The analgesic durations and anti-inflammatory effects of ketorolac and six ketorolac ester derivatives of formula (I) in rats are summarized in Table 7.

TABLE 7
The analgesic and anti-inflammatory durations** of
ketorolac and six ketorolac ester derivatives in
rats intraplantarly injected with carrageenin

| | | | Anti- |
|---|---|---|---|
| | Dose | Analgesic | inflammatory |
| Compound name | (μmole/Kg) | duration (h) | duration (h) |
| Ketorolac tromethamine | 24, 80, 240 | 6, 6, 8 | 8, 8, 8 |
| (in water) | | | |
| Ketorolac propyl ester | 80, 160, 240 | 6, 28, 54 | 6, 30, 54 |
| (in oil A)* | | | |
| Ketorolac propyl ester | 240 | 56, 56 | 54, 56 |
| (in oils B, C)* | | | |
| Ketorolac propyl ester | 240 | 56, 78 | 56, 78 |
| (in oils D, E)* | | | |
| Ketorolac tert-butyl ester | 240 | 56 | 56 |
| (in oil A)* | | | |
| Ketorolac benzyl ester | 240 | 56 | 56 |
| (in oil A)* | | | |
| Ketorolac pentyl ester | 240 | 56 | 56 |
| (in oil A)* | | | |
| Ketorolac heptyl ester | 240 | 74 | 74 |
| (in oil A)* | | | |
| Ketorolac decyl ester | 240 | 76 | 76 |
| (in oil A)* | | | |
| Ketorolac cetyl ester | 240 | 74 | 78 |
| (in oil A)* | | | |

*oil A, sesame oil; oil B, soybean oil; oil C, peanut oil; oil D, castor oil; and oil E, cotton seed oil.
**Ketorolac and six ketorolac ester derivatives of formula (I) were injected intramuscularly. Analgesic effect was evaluated by using the paw pressure test. Anti-inflammatory effect was evaluated by measuring the paw thickness.

### Pharmacological Ex. 4

### Evaluation of the In Vivo Analgesic Efficacy and Anti-Inflammatory Effect of Ketorolac Propyl Ester Prepared in 5 Oil Vehicles in Rats Intraplantarly Injected with Carrageenin

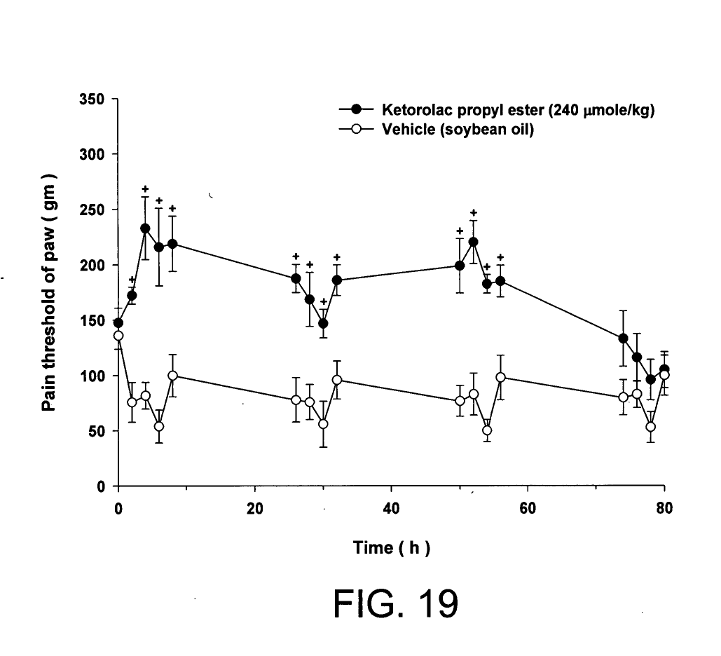

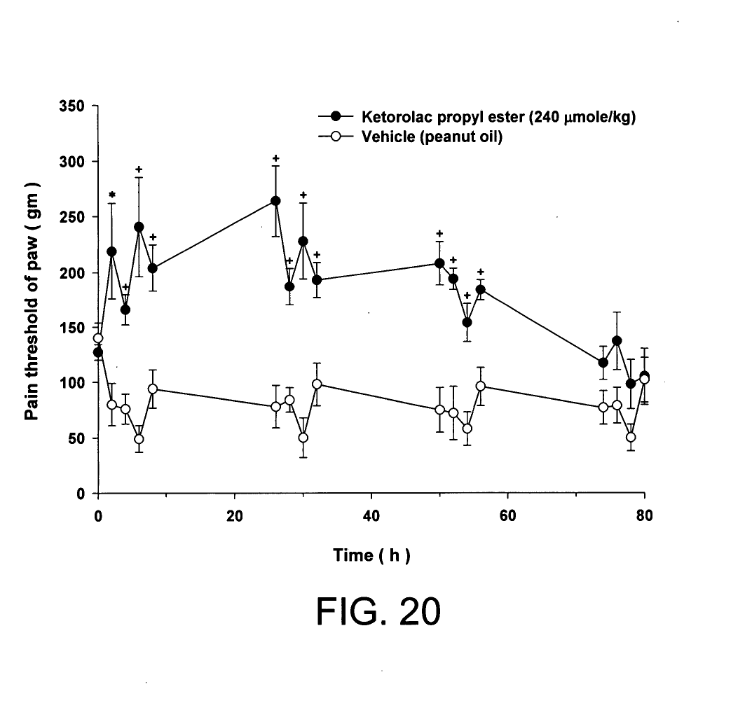

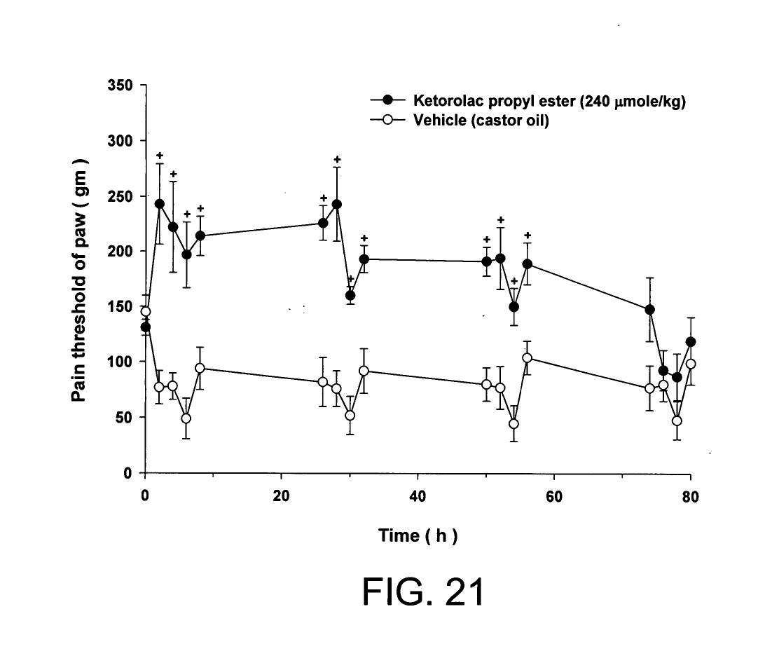

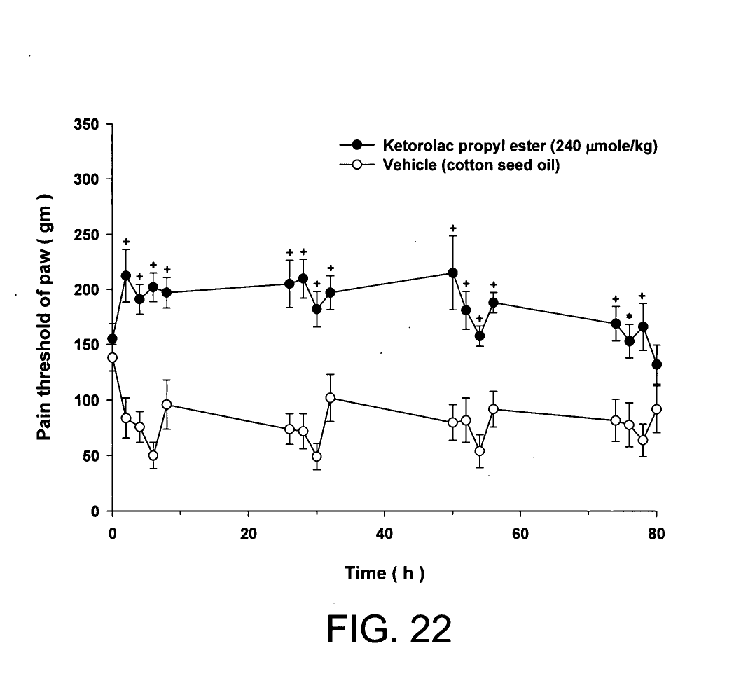

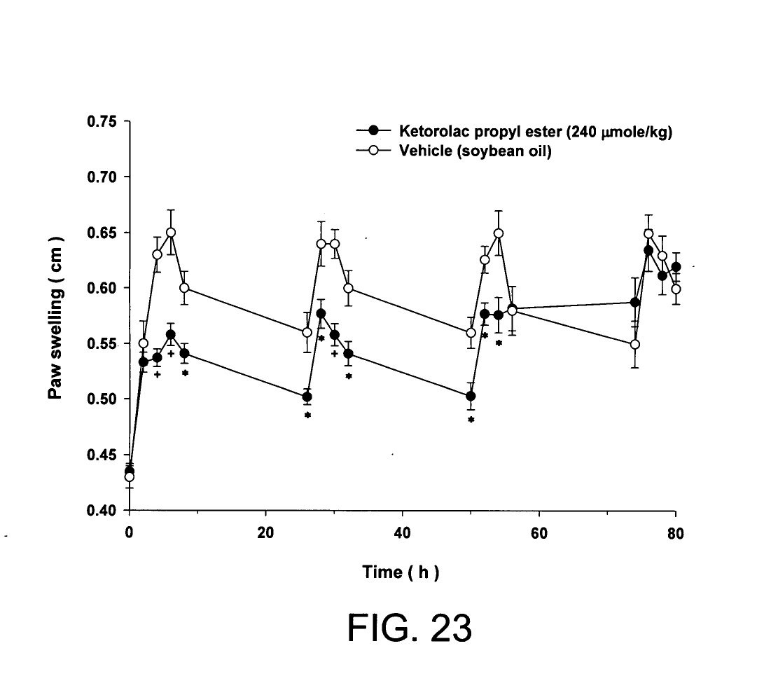

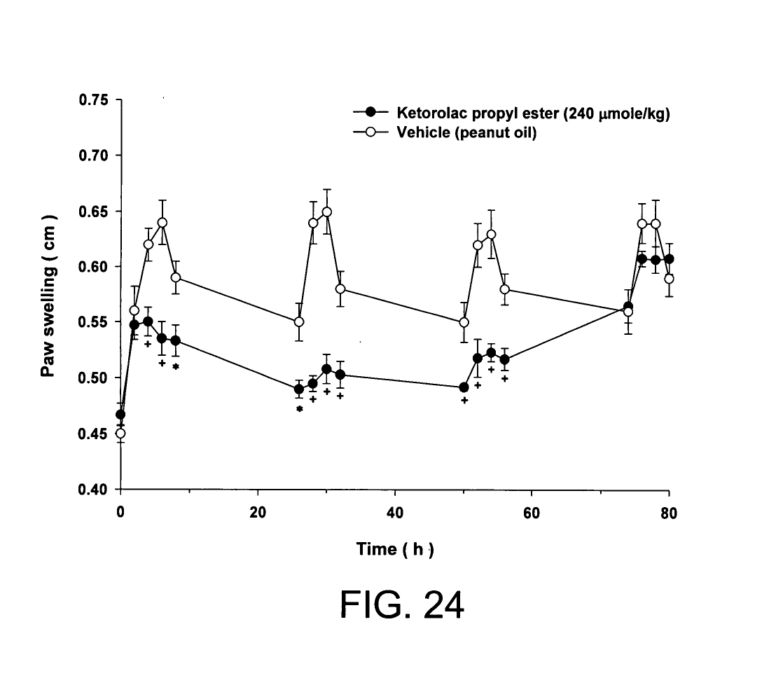

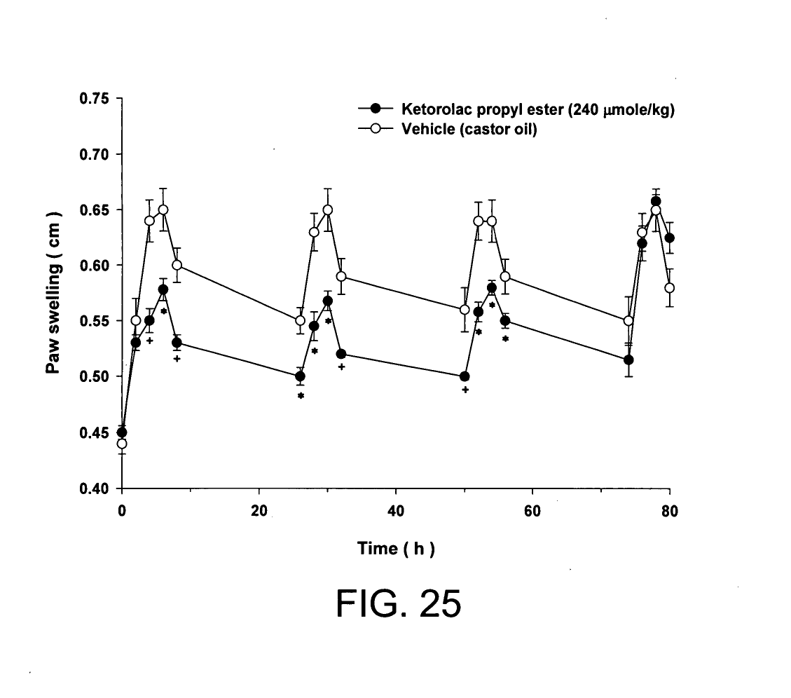

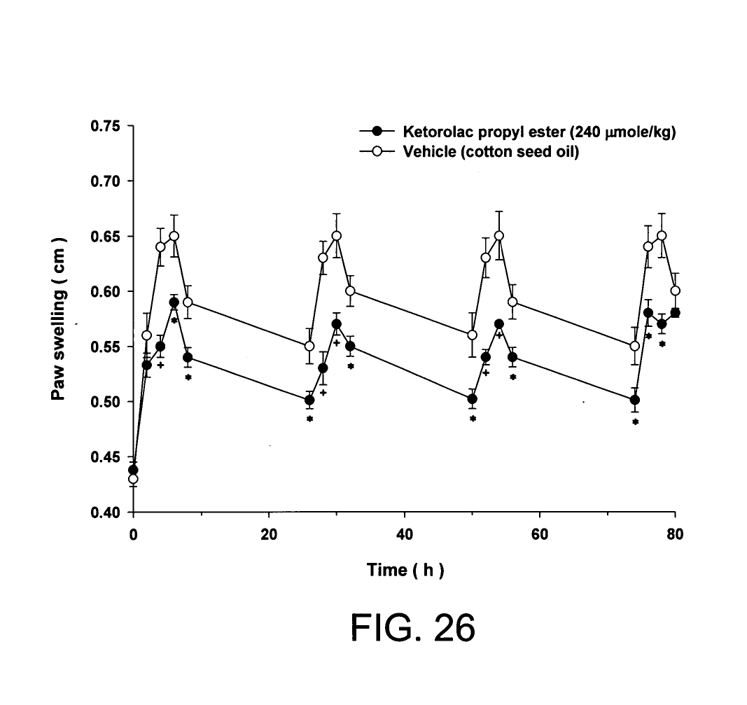

(1) Animal: male Sprague-Dawley rats (175-225 gm, 6 weeks old), n=6 in each group of each analgesic drug at each day of study.
(2) Study design: A 4-day study was carried out (see Pharmacological Example 2 described above). All rats received only one intramuscular injection of either a tested analgesic drug (ketorolac propyl ester prepared in four different oils) or the vehicle (4 different oils) at the start of study (day 1) and then received intraplantar injection of carrageenin at either day 1, 2, 3, or day 4. Each rat received only one injection of carrageenin. Subsequent to carrageenin injection, rats were observed for a period of 8 hours to determine the analgesic and anti-inflammatory effects of the tested analgesic drugs.
(3) Analgesic drug: ketorolac propyl ester, prepared in the following oils: sesame oil, soybean oil, castor oil, cotton seed oil, and peanut oil, dose in use: 240 μmole/Kg.
(4) Carrageenin injection: See Pharmacological Example 1 described above.
(5) Measurements of the pain threshold values of rat paw: See Pharmacological Example 1 described above.
(6) Measurement of paw swelling: See Pharmacological Example 1 described above.
(7) Statistics: The three-way analysis of variance with one-way repeated method followed by the Bonferroni test was used (see Pharmacological Example 2 described above).
(8) Results: All of the oil formulations of ketorolac propyl ester provided long-acting analgesic and anti-inflammatory effects (see Table 7, FIGS. 19-22 and FIGS. 23-26).

All patents and literature references cited in the present specification as well as the references described therein, are hereby incorporated by reference in their entirety. In case of conflict, the present description, including definitions, will prevail.

While the invention has been described in connection with specific embodiments thereof, it will be understood that it is capable of further modifications and this application is intended to cover any variations, uses, or adaptations of the invention following, in general, the principles of the invention and including such departures from the present customary practice within the art to which the invention pertains and as may be applied to the essential features hereinbefore set forth, and as follows in the scope of the appended claims.

What are claimed herein are:

1. An injectable long-acting analgesic composition comprising:
(a) a ketorolac ester derivative of formula (I): 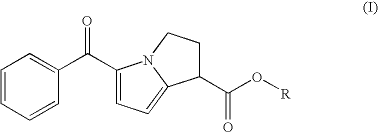
wherein
R is a straight-chain or branched saturated or unsaturated C₁-C₂₀ aliphatic group optionally substituted with a C₆-C₁₀ aryl group; and
(b) a pharmaceutically acceptable oil vehicle.

2. The composition of claim 1, wherein, in the ketorolac ester derivative of formula (I), R is a straight-chain or branched C₁-C₂₀ alkyl group optionally substituted with a C₆-C₁₀ aryl group.

3. The composition of claim 2, wherein, in the ketorolac ester derivative of formula (I), R is a straight-chain or branched C₁-C₂₀ alkyl group.

4. The composition of claim 3, wherein, in the ketorolac ester derivative of formula (I), R is a straight-chain C₃-C₁₆ alkyl group.

5. The composition of claim 3, wherein, in the ketorolac ester derivative of formula (I), R is a branched C₃-C₁₆ alkyl group.

6. The composition of claim 2, wherein, in the ketorolac ester derivative of formula (I), R is a straight-chain or branched C₁-C₂₀ alkyl group substituted with an aryl group selected from phenyl, naphthyl and tetrahydronaphthyl.

7. The composition of claim 6, wherein, in the ketorolac ester derivative of formula (I), R is a straight-chain or branched C₁-C₁₀ alkyl group substituted with a phenyl group.

8. The composition of claim 1, wherein, the ketorolac ester derivative of formula (I) is selected from the group consisting of ketorolac propyl ester, ketorolac tert-butyl ester, ketorolac pentyl ester, ketorolac hexyl ester, ketorolac heptyl ester, ketorolac decyl ester, ketorolac cetyl ester, and ketorolac benzyl ester.

9. The composition of claim 1, wherein, the oil vehicle is selected from the group consisting of sesame oil, castor oil, cotton seed oil, soybean oil, peanut oil, and combinations thereof.

10. The composition of claim 1, wherein, the composition is suitable for administration via intramuscular injection.

11. The composition of claim 1, wherein the composition is suitable for administration via subcutaneous injection.

12. The composition of claim 1, wherein the composition is able to provide a prolonged analgesia to a subject in need thereof.

13. The composition of claim 1, wherein the composition is able to provide a prolonged anti-inflammatory effect to a subject in need thereof.

14. A method for providing a prolonged analgesia to a subject, comprising administering an effective amount of the composition of claim 1 to a subject in need of such treatment.

15. The method of claim 14, wherein the composition of claim 1 is administered via intramuscular injection.

16. The method of claim 14, wherein the composition of claim 1 is administered via subcutaneous injection.

17. A method for providing a prolonged anti-inflammatory effect to a subject, comprising administering an effective amount of the composition of claim 1 to a subject in need of such treatment.

18. The method of claim 17, wherein the composition of claim 1 is administered via intramuscular injection.

19. The method of claim 17, wherein the composition of claim 1 is administered via subcutaneous injection.
